(19) United States
(12) Patent Application Publication
(10) Pub. No.: US 2024/0271223 A1
(43) Pub. Date: Aug. 15, 2024

(54) BIOMARKERS FOR A THERAPY COMPRISING AN ANGIOGENESIS INHIBITOR

(71) Applicant: Eisai R&D Management Co., Ltd., Bunkyo-ku, Tokyo (JP)

(72) Inventors: Megumi KURONISHI, Tsukuba (JP); Yu Kato, Tsukuba (JP)

(21) Appl. No.: 18/569,317

(22) PCT Filed: Apr. 7, 2022
(86) PCT No.: PCT/JP2022/017262

§ 371 Date: Dec. 12, 2023

Related U.S. Application Data
(60) Provisional application No. 63/223,153, filed on Jul. 19, 2021.

(51) Int. Cl. C12Q 1/6886; A61K 31/47; A61P 35/00

(52) U.S. Cl. CPC C12Q 1/6886; A61K 31/47; A61P 35/00; G01N 2800/52

(57) ABSTRACT

Biomarkers are provided that predict whether a human subject having a tumor is in need of a therapy comprising an angiogenesis inhibitor (e.g. lenvatinib or a pharmaceutically acceptable salt thereof, such as lenvatinib mesylate). The biomarkers, compositions, and methods described herein are useful in selecting appropriate treatment modalities for and treating a subject having a tumor.

### TECHNICAL FIELD

The present invention relates generally to biomarkers and cancer treatment.

### BACKGROUND ART

A number of kinase inhibitors have been developed as antitumor agents. For example, a group of compounds having inhibitory activity against receptor tyrosine kinases, such as vascular endothelial growth factor receptor (VEGFR), are known to inhibit angiogenesis and are regarded as a new class of antitumor agents.

An angiogenesis inhibitor lenvatinib mesylate (also known as E7080) is an oral tyrosine kinase inhibitor targeting VEGFR1-3, fibroblast growth factor receptor (FGFR) 1-4, rearranged during transfection receptor (RET), KIT, and platelet-derived growth factor receptor (PDGFR). Lenvatinib mesylate has been approved as LENVIMA® by U.S. Food and Drug Administration for the treatment of patients with unresectable differentiated thyroid cancer, advanced renal cell carcinoma (in combination with everolimus), unresectable hepatocellular carcinoma (HCC), and endometrial carcinoma (in combination with pembrolizumab). Other tumor types have been approved, submitted for approval or are currently under phase 2 or 3 clinical studies, including thymic carcinoma, melanoma, non-small cell lung cancer, head and neck squamous cell carcinoma, ovarian cancer, urothelial carcinoma, triple negative breast cancer, biliary tract cancer, glioblastoma, pancreatic cancer and colorectal cancer are ongoing.

Unfortunately, most anti-tumor treatments are associated with undesirable side effects, such as profound nausea, vomiting, or severe fatigue. Also, while anti-tumor treatments have been successful, they do not produce significant clinical responses in all patients who receive them, resulting in undesirable side effects, delays, and costs associated with ineffective treatment. Therefore, biomarkers that can be used to predict the response of a subject to an antitumor agent, prior to administration thereof are greatly needed.

Yamamoto et al. Vasc Cell., 6:18 (2014) reports that human xenograft tumors sensitive to lenvatinib showed higher microvessel density (MVD) score, as calculated by CD31-positive blood vessel number divided with tumor area in immunohistochemistry (IHC), than tumors relatively resistant to lenvatinib.

Matsuki et al. Cancer Med., 7(6), pp. 2641-2653 (2018) reports that administration of lenvatinib reduced tumor microvessel density in PLC/PRF/5 xenograft HCC model and two HCC patient-derived xenograft models.

Although MVD score as calculated by IHC (“MVD (IHC)”) is associated with antitumor effect of lenvatinib, obtaining tumor tissue with a mass sufficient for an IHC analysis and conducting such analysis are not always clinically practical.

### SUMMARY OF INVENTION

The present application is based, at least in part, on the identification of biomarkers that correlate with MVD (IHC). More specifically, a score calculated from the sum of mRNA expression levels of selected genes comprising two or more of kinase insert domain receptor (KDR), cadherin 5 (CDH5), endoglin (ENG), TEK receptor tyrosine kinase (TEK), protocadherin 12 (PCDH12) or SPARC like 1 (SPARCL1) in a tumor tissue obtained from the human subject (“gene score”) is found to correlate with MVD (IHC). Thus, the biomarkers described herein are useful, for example, in identifying, stratifying, and/or selecting a patient or a subset of patients having a tumor that could benefit from a therapy comprising an angiogenesis inhibitor (e.g., lenvatinib or a pharmaceutically acceptable salt thereof, such as lenvatinib mesylate).

In one aspect, the disclosure provides a method of assessing MVD by novel biomarkers. The method involves assaying a tumor tissue sample obtained from the human subject and determining a gene score calculated from the sum of mRNA expression levels of selected genes comprising two or more of KDR, CDH5, ENG, TEK, PCDH12 or SPARCL1 in the tumor tissue.

In a second aspect, the disclosure features a method of selecting a human subject having a tumor for administration with a therapy comprising an angiogenesis inhibitor (e.g., lenvatinib or a pharmaceutically acceptable salt thereof, such as lenvatinib mesylate). The method comprises assaying a tumor tissue sample obtained from the human subject for the mRNA expression level of selected genes comprising two or more of KDR, CDH5, ENG, TEK, PCDH12 or SPARCL1. If it is determined that a gene score calculated based on the mRNA expression level of the selected genes is high, as compared to a control, the human subject is selected for administration of the therapy. In certain embodiments, the method further comprises administering the therapy to the human subject.

In a third aspect, the disclosure provides a method of treating a human subject having a tumor. The method involves providing a tumor tissue sample obtained from a human subject having a tumor before the treatment; measuring, in the tumor tissue sample, mRNA expression level of selected genes comprising two or more of KDR, CDH5, ENG, TEK, PCDH12 or SPARCL1; calculating a gene score based on the mRNA expression level that is high as compared to a control; and administering to the human subject a therapeutically effective amount of an angiogenesis inhibitor (e.g., lenvatinib or a pharmaceutically acceptable salt thereof, such as lenvatinib mesylate).

In a fourth aspect, the disclosure provides a method of treating a human subject having a tumor. The method involves administering to the human subject that has a tumor a therapeutically effective amount of an angiogenesis inhibitor (e.g., lenvatinib or a pharmaceutically acceptable salt thereof, such as lenvatinib mesylate), wherein the human subject has been identified as having a gene score based on the mRNA expression level of selected genes comprising two or more of KDR, CDH5, ENG, TEK, PCDH12 or SPARCL1 that is high as compared to a control. In certain embodiments, the human subject has been identified as having a high mRNA expression level of selected genes comprising two or more of KDR, CDH5, ENG, TEK, PCDH12 or SPARCL1 in a tumor tissue sample obtained from the human subject.

In a fifth aspect, the disclosure features a method of treating a human subject having a tumor. The method involves administering the human subject with a therapy comprising an angiogenesis inhibitor (e.g., lenvatinib or a pharmaceutically acceptable salt thereof, such as lenvatinib mesylate), wherein the human subject has been previously determined to have a gene score based on the mRNA expression level of selected genes comprising two or more of KDR, CDH5, ENG, TEK, PCDH12 or SPARCL1 in a tumor tissue sample obtained from the human subject that is higher than a control.

In a sixth aspect, the disclosure features a kit for assessing microvessel density of a tumor tissue sample by measuring mRNA expression level of selected genes comprising two or more of KDR, CDH5, ENG, TEK, PCDH12 or SPARCL1 in the tumor tissue sample. In certain embodiments, the kit includes one or more agents to quantify mRNA expression level of selected genes comprising two or more of KDR, CDH5, ENG, TEK, PCDH12 or SPARCL1. In certain embodiments, such agents include one or more primers, probes or fluorescently bar-coded oligonucleotide probes for hybridization to RNA. In certain embodiments, the kit optionally includes one or more concentration standards, one or more buffers (e.g., wash buffers), one or more diluents (e.g., assay and/or calibration diluents), and instructions for calculating a gene score based on the mRNA expression level of selected genes comprising two or more of KDR, CDH5, ENG, TEK, PCDH12 or SPARCL1.

The following embodiments are envisaged for all of the above aspects.

In one embodiment the angiogenesis inhibitor is lenvatinib or a pharmaceutically acceptable salt thereof.

In one embodiment the lenvatinib or a pharmaceutically acceptable salt thereof is lenvatinib mesylate.

In one embodiment, the tumor is not gastrointestinal cancer.

In one embodiment, the tumor is thyroid cancer, renal cell carcinoma, breast cancer, endometrial cancer, glioblastoma, head and neck cancer, non-small cell lung cancer, ovarian cancer, pancreatic cancer, cervical cancer, hepatocellular carcinoma, thymic carcinoma, melanoma, urothelial carcinoma, colorectal cancer or biliary tract cancer.

In one embodiment, the tumor is thyroid cancer, renal cell carcinoma, breast cancer, endometrial cancer, glioblastoma, head and neck cancer, non-small cell lung cancer, ovarian cancer, pancreatic cancer, cervical cancer or melanoma.

In some embodiments, the gene score is proportional to the average of logarithm-transformed mRNA expression level of the selected genes without weighting coefficient of each gene.

In some embodiments, the gene score is proportional to the average of logarithm-transformed mRNA expression level of the selected genes with weighting coefficients optimized for each gene. Methods for optimizing weighting coefficients for each gene are well known to those skilled in the art. For example, Ayers et al. J Clin Invest. 127(8), pp.2930-2940 (2017) discloses a method for optimizing weighting coefficients by regression analysis.

In some embodiments, the control is a pre-established cut-off value. In one embodiment, the pre-established cut-off value is a gene score that is determined based on receiver operating characteristic (ROC) analysis or percentile analysis predicting tumor response with a higher positive predictive value compared to no cut-off, and wherein a gene score equal to or below the pre-established cut-off value is a low gene score and a value higher than the pre-established cut-off value is a high gene score. The tumor response is an objective response rate (ORR) or % of maximum tumor shrinkage. In another embodiment, the pre-established cut-off value is a gene score that is determined based on simulation models or percentile analysis predicting survival, and wherein a gene score equal to or below the pre-established cut-off value is a low gene score and a value higher than the pre-established cut-off value is a high gene score. In this context, survival is progression free survival (PFS) or overall survival (OS).

In some embodiments, the method further includes communicating the test results to the human subject's health care provider. In certain embodiments, the method further includes modifying the human subject's medical record to indicate that the human subject is in need of or not in need of a therapy comprising an angiogenesis inhibitor (e.g., lenvatinib or a pharmaceutically acceptable salt thereof, such as lenvatinib mesylate). In specific embodiments, the record is created on a computer readable medium. In certain embodiments, the method further includes prescribing a therapy comprising an angiogenesis inhibitor (e.g., lenvatinib or a pharmaceutically acceptable salt thereof, such as lenvatinib mesylate) for the human subject if the gene score calculated based on the mRNA expression level of selected genes comprising two or more of KDR, CDH5, ENG, TEK, PCDH12 or SPARCL1 is predictive that the human subject is in need of a therapy comprising an angiogenesis inhibitor (e.g., lenvatinib or a pharmaceutically acceptable salt thereof, such as lenvatinib mesylate). In certain embodiments, the method further includes prescribing a therapy not comprising an angiogenesis inhibitor (e.g., lenvatinib or a pharmaceutically acceptable salt thereof, such as lenvatinib mesylate) for the human subject if the gene score calculated based on the mRNA expression level of selected genes comprising two or more of KDR, CDH5, ENG, TEK, PCDH12 or SPARCL1 is predictive that the human subject is not in need of a therapy comprising an angiogenesis inhibitor (e.g., lenvatinib or a pharmaceutically acceptable salt thereof, such as lenvatinib mesylate). In some embodiments, the method further includes administering to the human subject a therapy comprising an angiogenesis inhibitor (e.g., lenvatinib or a pharmaceutically acceptable salt thereof, such as lenvatinib mesylate) if the gene score calculated based on the mRNA expression level of selected genes comprising two or more of KDR, CDH5, ENG, TEK, PCDH12 or SPARCL1 is predictive that the human subject is in need of a therapy comprising an angiogenesis inhibitor (e.g., lenvatinib or a pharmaceutically acceptable salt thereof, such as lenvatinib mesylate). In some embodiments, the method further includes administering to the human subject a therapy that does not comprise an angiogenesis inhibitor (e.g., lenvatinib or a pharmaceutically acceptable salt thereof, such as lenvatinib mesylate) if the gene score calculated based on the mRNA expression level of selected genes comprising two or more of KDR, CDH5, ENG, TEK, PCDH12 or SPARCL1 is predictive that the human subject is not in need of a therapy comprising an angiogenesis inhibitor (e.g., lenvatinib or a pharmaceutically acceptable salt thereof, such as lenvatinib mesylate).

Methods for quantifying the mRNA expression level of a gene are well known to those skilled in the art. In one embodiment, such method is selected from Northern blot or dot blot analysis, nucleic acid microarray (e.g., oligonucleotide arrays or gene chips), in situ hybridization (e.g., quantitative in situ hybridization), reverse transcriptase-polymerase chain reaction (RT-PCR; e.g., quantitative RT-PCR), digital PCR, molecule quantification system using molecular barcodes such as nCounter® Analysis System (NanoString Technologies), next generation sequencing (NGS) such as RNA sequencing (RNA-seq) or single cell RNA sequencing (scRNA-seq). In one embodiment, such method is RNA-seq or scRNA-seq. In one embodiment, such method is RNA-seq.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF DRAWINGS

FIG. 1 is violin plots showing expression of six MVD-related genes in single cell RNA-seq analysis of Hepa 1-6 mouse syngeneic tumor in endothelial cluster and in clusters other than endothelial cluster. EC: endothelial cluster.

FIG. 2 is a scatterplot showing correlation of gene score and MVD (IHC_{CD31}) across 12 syngeneic tumor models. Mean values of each model are shown. Hepa: Hepa 1-6, BNL: BNL 1ME A.7R.1, CT26: CT26.WT.

FIGS. 3a-3c are boxplots showing mean MVD (IHC_{CD31}) (FIG. 3a), mean gene score (FIG. 3b), and antitumor activity per model in three lenvatinib sensitive models and other nine models (FIG. 3c). Difference between two groups is statistically significant: (a) p<0.001 (t-test), (b) p<0.01 (t-test), (c) p<0.05 (t-test).

FIG. 4 is a boxplot showing antitumor activity of lenvatinib in 12 syngeneic tumor models in high- (left) and low-gene score (right) groups. High- and low-gene scores are divided by cut-off value (3.69). * represents p<0.05 (t-test).

FIG. 5 depicts scatterplots of gene score and MVD (IHC_{CD31}) using all 24 human tumor samples (left) and 21 human tumor samples in which three gastrointestinal cancer samples are excluded (right).

FIG. 6 is scatter plot of gene score and MVD (IHC_{CD34}) using 47 human tumor samples.

FIG. 7 is box plot of gene score of 12 mouse syngeneic tumor models. Hepa: Hepa 1-6; BNL: BNL 1ME A.7R.1; CT26: CT26.WT; NT: no-treatment; AFL: Aflibercept; LEN: lenvatinib. * represents p<0.05 vs NT (t-test).

### DESCRIPTION OF EMBODIMENTS

This disclosure provides methods for identifying a human subject (such as a human patient) having a tumor in need of a therapy comprising an angiogenesis inhibitor (e.g., lenvatinib or a pharmaceutically acceptable salt thereof, such as lenvatinib mesylate). The disclosure provides KDR, CDH5, ENG, TEK, PCDH12 and SPARCL1 as predictive biomarkers to identify those human subjects having a tumor for whom administering a therapy comprising an angiogenesis inhibitor (e.g., lenvatinib or a pharmaceutically acceptable salt thereof, such as lenvatinib mesylate) is recommended. The biomarkers and methods described herein are useful in selecting appropriate therapeutic modalities (e.g., therapy comprising an angiogenesis inhibitor (e.g., lenvatinib or a pharmaceutically acceptable salt thereof, such as lenvatinib mesylate) or an alternative tumor therapy) for human subjects having a tumor. Furthermore, this application provides methods of assessing MVD by quantifying mRNA of biomarker genes.

### Definitions

The term “a human subject in need of a therapy” means a human subject for whom a therapy is recommendable as a preferable treatment.

The term “mRNA expression level” means a determined level of mRNA expression of a gene. The mRNA expression level of a gene can be measured directly, for example, by obtaining a signal, the signal intensity of which correlates with the amount of mRNA transcript of the gene. The expression level can also be obtained by competitive reaction with regard to the reference sample.

The term “angiogenesis inhibitor” means any substance having an angiogenesis inhibitory activity. Examples of the angiogenesis inhibitor include a VEGF receptor inhibitor, an FGF receptor inhibitor, a PDGF receptor inhibitor, a RET kinase inhibitor, a KIT kinase inhibitor, an epithelial growth factor (EGF) inhibitor, an integrin inhibitor, a matrix metalloprotease inhibitor and an endogenous inhibitor. In one embodiment, angiogenesis inhibitor is a VEGF receptor inhibitor, which includes, but not limited to, lenvatinib, sorafenib, sunitinib, pazopanib, cabozantinib, regorafenib, axitinib, ponatinib, vandetanib, tivozanib, bevacizumab, ramucirumab, ranibizumab and VEGF-Trap (e.g., aflibercept).

The term “lenvatinib” refers to 4-(3-chloro-4-(cyclopropylaminocarbonyl)aminophenoxy)-7-methoxy-6-quinolinecarb oxamide. This compound is disclosed in Example 368 (see, column 270) of U.S. Pat. No. 7,253,286. U.S. Pat. No. 7,253,286 is incorporated by reference in its entirety herein. The term “lenvatinib compound” refers to “lenvatinib or a pharmaceutically acceptable salt thereof.” An example of a pharmaceutically acceptable salt of lenvatinib is lenvatinib mesylate. Lenvatinib mesylate is also referred to as E7080. Lenvatinib mesylate has been approved as LENVIMA® by the U.S. Food and Drug Administration.

The term “pharmaceutically acceptable salt” is not particularly restricted as to the type of salt. Examples of such salts include, but are not limited to, inorganic acid addition salt such as hydrochloric acid salt, sulfuric acid salt, carbonic acid salt, bicarbonate salt, hydrobromic acid salt and hydriodic acid salt; organic carboxylic acid addition salt such as acetic acid salt, maleic acid salt, lactic acid salt, tartaric acid salt and trifluoroacetic acid salt; organic sulfonic acid addition salt such as methane-sulfonic acid salt, hydroxymethanesulfonic acid salt, hydroxyethanesulfonic acid salt, benzenesulfonic acid salt, toluenesulfonic acid salt and taurine salt; amine addition salt such as trimethylamine salt, triethylamine salt, pyridine salt, procaine salt, picoline salt, dicyclohexylamine salt, N,N′-dibenzylethylenediamine salt, N-methylglucamine salt, diethanolamine salt, triethanolamine salt, tris(hydroxymethylamino)methane salt and phenethylbenzylamine salt; and amino acid addition salt such as arginine salt, lysine salt, serine salt, glycine salt, aspartic acid salt and glutamic acid salt. In one embodiment, the pharmaceutically acceptable salt is a methanesulfonic acid salt (“mesylate”). The methanesulfonic acid salt form (i.e., the mesylate) of 4-(3-chloro-4-(cyclopropylaminocarbonyl)aminophenoxy)-7-methoxy-6-quinolinecarb oxamide is disclosed in U.S. Pat. No. 7,612,208, which is incorporated by reference herein in its entirety.

The term “responds/responsive to a therapy” means that the human subject administered with the therapy shows a positive response to the therapy provided. Non-limiting examples of such a positive response are: a decrease in tumor size, a decrease in metastasis of a tumor, or an increased period of survival after treatment.

A higher value of a gene score compared to a control is indicative/predictive that a human subject is in need of a therapy comprising an angiogenesis inhibitor (e.g., a lenvatinib compound, such as lenvatinib mesylate).

In certain embodiments, a human subject is determined to respond to a therapy comprising an angiogenesis inhibitor (e.g., a lenvatinib compound, such as lenvatinib mesylate), if the subject shows a partial response following treatment with the therapy. “Partial Response” means at least 30% decrease in the sum of the longest diameter (LD) of target lesions, taking as reference the baseline summed LD. In some embodiments, a human subject is determined to respond to a therapy comprising an angiogenesis inhibitor (e.g., a lenvatinib compound, such as lenvatinib mesylate), if the human subject shows tumor shrinkage post-treatment with the therapy. “% of maximum tumor shrinkage” (MTS) means percent change of sum of diameters of target lesions, taking as reference the baseline sum diameters. In other embodiments, a human subject is determined to respond to a therapy comprising an angiogenesis inhibitor (e.g., a lenvatinib compound, such as lenvatinib mesylate), if the subject shows overall survival. “Overall Survival” (OS) refers to the time from randomization until death from any cause. “Randomization” means randomization of a patient into a test group or a control group when therapy plan for a patient is determined. In some embodiments, a subject is determined to respond to a therapy comprising an angiogenesis inhibitor (e.g., a lenvatinib compound, such as lenvatinib mesylate), if the subject shows both overall survival and tumor shrinkage. In other embodiments, a human subject is determined to respond to a therapy comprising an angiogenesis inhibitor (e.g., a lenvatinib compound, such as lenvatinib mesylate), if the human subject shows progression free survival. “Progression Free Survival” (PFS) refers to the time from the date of randomization to the date of first documentation of disease progression or death, whichever occurs first. In some embodiments, a human subject is determined to respond to a therapy comprising an angiogenesis inhibitor (e.g., a lenvatinib compound, such as lenvatinib mesylate), if the subject shows both progression free survival and tumor shrinkage.

The treatment comprising administering an angiogenesis inhibitor (e.g., lenvatinib or a pharmaceutically acceptable salt thereof, such as lenvatinib mesylate) described herein may further comprise concomitant administration of one or more drugs to a human subject. Such one or more drugs can be administered simultaneously, substantially simultaneously, or sequentially with the angiogenesis inhibitor. In some cases, two or more drugs may be formulated together (e.g., into a single tablet or capsule). In other cases, the two or more drugs are not co-formulated (e.g., they are administered as separate tablets or capsules).

In certain embodiments, a lenvatinib compound (e.g., lenvatinib mesylate) can be administered to a subject of the present invention orally once daily at a dosage of 8, 12, 18, 20 or 24 mg (each calculated as lenvatinib free base).

### Controls

In the methods described herein, the genes from which the gene score is calculated comprise at least two genes selected from the group consisting of KDR, CDH5, ENG, TEK, PCDH12 or SPARCL1. The genes can further comprise one or more additional genes other than the aforementioned six genes.

As described above, the methods described herein can involve, calculating a gene score of a tumor tissue sample obtained from a human subject having a tumor, wherein the gene score, compared to a control, predicts that the human subject is in need of (or can benefit from) a treatment comprising an angiogenesis inhibitor (e.g., a lenvatinib compound, such as lenvatinib mesylate). In certain embodiments, when the gene score in a biological sample from a subject having a tumor is higher than the control, the subject is identified as in need of a therapy comprising an angiogenesis inhibitor (e.g., a lenvatinib compound, such as lenvatinib mesylate). In this context, the term “control” includes a tumor tissue sample (e.g., from the same tissue) obtained from a human subject who is not in need of a therapy comprising an angiogenesis inhibitor (e.g., a lenvatinib compound, such as lenvatinib mesylate). Such subject who is not in need of a therapy comprising an angiogenesis inhibitor (e.g., a lenvatinib compound, such as lenvatinib mesylate) may include a human subject who is predicted to respond to the therapy but such response to the therapy is not significantly better than a predicted response to a therapy with other drugs. The term “control” also includes a sample (e.g., from the same tumor tissue) obtained in the past from a human subject who is known to be not in need of a therapy comprising an angiogenesis inhibitor (e.g., a lenvatinib compound, such as lenvatinib mesylate) and used as a reference for future comparisons to test samples taken from human subjects for which necessity for the therapy is to be predicted.

In some embodiments, a “positive control” may be used instead of a “control.” The “positive control” gene score of a tumor tissue may alternatively be pre-established by an analysis of one or more human subjects that have been identified as in need of a therapy comprising an angiogenesis inhibitor (e.g., a lenvatinib compound, such as lenvatinib mesylate). This pre-established reference value (which may be an average or median expression level taken from multiple human subjects that have been identified as in need of a therapy) may then be used as the “positive control” gene score in the comparison with the test sample. In such a comparison, the subject is predicted to be in need of a therapy comprising an angiogenesis inhibitor (e.g., a lenvatinib compound, such as lenvatinib mesylate) if the gene score being analyzed is the same as, or comparable to (at least 85% but less than 100% of), the pre-established positive control reference.

In certain embodiments, the “control” is a pre-determined cut-off value.

### Cut-Off Values

In some embodiments, the methods described herein include determining if the gene score falls above or below a predetermined cut-off value.

In accordance with the methods and compositions described herein, a reference gene score is identified as a cut-off value, above or below of which is predictive of necessity for a therapy comprising an angiogenesis inhibitor (e.g. a lenvatinib compound, such as lenvatinib mesylate). Some cut-off values are not absolute in that clinical correlations can still remain significant over a range of values on either side of the cutoff; however, it is possible to select an optimal cut-off value gene score for a particular sample type. Cut-off values determined for use in the methods described herein can be compared with, e.g., published ranges of gene score but can be individualized to the methodology used and patient population. It is understood that improvements in optimal cut-off values could be determined depending on the sophistication of statistical methods used and on the number and source of samples used to determine reference level values for the different sample types. Therefore, established cut-off values can be adjusted up or down, on the basis of periodic re-evaluations or changes in methodology or population distribution.

The reference gene score can be determined by a variety of methods. The reference gene score can be determined by comparison of the gene score of interest in, e.g., populations of subjects (e.g., patients) that are in need of a therapy comprising an angiogenesis inhibitor (e.g., a lenvatinib compound, such as lenvatinib mesylate) or not in need of a therapy comprising an angiogenesis inhibitor (e.g., a lenvatinib compound, such as lenvatinib mesylate). This can be accomplished, for example, by histogram analysis, in which an entire cohort of patients are graphically presented, wherein a first axis represents the gene score and a second axis represents the number of subjects in the cohort whose sample contain one or more gene score. Determination of the reference gene score can then be made based on a gene score which best distinguishes these separate groups. The reference gene score can be a single number, equally applicable to every subject, or the reference gene score can vary, according to specific subpopulations of subjects. For example, older subjects can have a different reference gene score than younger subjects for the same cancer. In addition, a subject with more advanced disease (e.g., an advanced or metastatic carcinoma) can have a different reference value than one with a milder form of the disease.

The pre-established cut-off value can be a gene score that is determined based on receiver operating characteristic (ROC) analysis. ROC curves are used to determine a cut-off value for a clinical test. Consider the situation where there are two groups of patients and by using an established standard technique one group is known to be in need of a therapy comprising an angiogenesis inhibitor (e.g., a lenvatinib compound, such as lenvatinib mesylate), and the other is known to not in need of a therapy comprising an angiogenesis inhibitor (e.g., a lenvatinib compound, such as lenvatinib mesylate). A measurement using a tumor tissue sample from all members of the two groups is used to test for the necessity for a therapy comprising an angiogenesis inhibitor (e.g., a lenvatinib compound, such as lenvatinib mesylate). The test will find some, but not all, subjects that are in need of a therapy comprising an angiogenesis inhibitor (e.g., a lenvatinib compound, such as lenvatinib mesylate). The ratio of the subjects in need of the therapy found by the test to the total number of the subjects in need of the therapy (known by the established standard technique) is the true positive rate (also known as sensitivity). The test will find some, but not all, the human subjects not in need of a therapy comprising an angiogenesis inhibitor (e.g., a lenvatinib compound, such as lenvatinib mesylate). The ratio of the subjects not in need of the therapy found by the test to the total number of the subjects not in need of the therapy (known by the established standard technique) is the true negative rate (also known as specificity). The hope is that the ROC curve analysis of the test above will find a cut-off value that will minimize the number of false positives and false negatives. A ROC is a graphical plot which illustrates the performance of a binary class stratifier system as its discrimination threshold is varied. It is created by plotting the fraction of true positives out of the positives versus the fraction of false positives out of the negatives, at various threshold settings.

In one embodiment, the gene score is determined based on ROC analysis predicting tumor response with a positive predictive value, wherein a gene score equal to or below the pre-established cut-off value is a low gene score and a value higher than the pre-established cut-off value is a high gene score. The positive predictive value is the proportion of positive test results that are true positives; it reflects the probability that a positive test reflects the underlying condition being tested for. Methods of constructing ROC curves and determining positive predictive values are well known in the art. In certain embodiments, tumor response is an objective response rate (ORR), a clinical benefit rate (CBR) or % of maximum tumor shrinkage.

In another embodiment, the pre-established cut-off value can be a gene score that is determined based on simulation models predicting survival, and wherein a gene score equal to or below the pre-established cut-off value is a low gene score and a value higher than the pre-established cut-off value is a high gene score. In some embodiments, survival is progression free survival (PFS). In other embodiments, survival is overall survival (OS).

In all of these embodiments, a gene score equal to or below the pre-established cut-off value is a low gene score and a value higher than the pre-established cut-off value is a high gene score. In this context “about” means ±10%.

### Tumor Tissue Samples

Methods for obtaining and/or storing tumor tissue samples that preserve the activity or integrity of molecules (e.g., nucleic acids or proteins) in the sample are well known to those skilled in the art. For example, a tumor tissue sample can be further contacted with one or more additional agents such as buffers and/or inhibitors, including one or more of nuclease, protease, and phosphatase inhibitors, which preserve or minimize changes in the molecules (e.g., nucleic acids or proteins) in the sample. Such inhibitors include, for example, chelators such as ethylenediamine tetraacetic acid (EDTA), ethylene glycol bis(P-aminoethyl ether) N,N,N1,Nl-tetraacetic acid (EGTA), protease inhibitors such as phenylmethylsulfonyl fluoride (PMSF), aprotinin, leupeptin, antipain, and the like, and phosphatase inhibitors such as phosphate, sodium fluoride, vanadate, and the like. Suitable buffers and conditions for isolating molecules are well known to those skilled in the art and can be varied depending, for example, on the type of molecule in the sample to be characterized (see, for example, Ausubel et al. Current Protocols in Molecular Biology (Supplement 47), John Wiley & Sons, New York (1999); Harlow and Lane, Antibodies: A Laboratory Manual (Cold Spring Harbor Laboratory Press (1988); Harlow and Lane, Using Antibodies: A Laboratory Manual, Cold Spring Harbor Press (1999); Tietz Textbook of Clinical Chemistry, 3rd ed. Burtis and Ashwood, eds. W. B. Saunders, Philadelphia, (1999)). A sample also can be processed to eliminate or minimize the presence of interfering substances. For example, a biological sample can be fractionated or purified to remove one or more materials that are not of interest. Methods of fractionating or purifying a tumor tissue sample include, but are not limited to, chromatographic methods such as liquid chromatography, ion-exchange chromatography, size-exclusion chromatography, or affinity chromatography.

### Creating a Response Profile

The methods described herein can also be used to generate a response profile for a human subject having a tumor about a therapy comprising an angiogenesis inhibitor (e.g., lenvatinib compound, such as lenvatinib mesylate). The profile can include, e.g., information that indicates the gene score before the treatment with an angiogenesis inhibitor (e.g., lenvatinib or a pharmaceutically acceptable salt thereof, such as lenvatinib mesylate); and/or the histological analysis of the tumor tissue obtained from the human subject. The resultant information (lenvatinib therapy response profile) can be used for predicting that a subject (e.g., a human patient) having a tumor is in need of a therapy comprising an angiogenesis inhibitor (e.g., lenvatinib compound, such as lenvatinib mesylate).

It is understood that an angiogenesis inhibitor (e.g., lenvatinib compound, such as lenvatinib mesylate) response profile can be in electronic form (e.g., an electronic patient record stored on a computer or other electronic (computer-readable) media such as a DVD, CD, or floppy disk) or written form. The angiogenesis inhibitor (e.g., lenvatinib compound, such as lenvatinib mesylate) response profile can also include information for several (e.g., two, three, four, five, 10, 20, 30, 50, or 100 or more) human subjects. Such multi-subject response profiles can be used, e.g., in analyses (e.g., statistical analyses) of particular characteristics of subject cohorts.

Responsiveness of a human subject to a therapy comprising an angiogenesis inhibitor (e.g., a lenvatinib compound, such as lenvatinib mesylate) can be classified in several ways and classification is dependent on the subject's disease, the severity of the disease, and the particular medicament the subject is administered. In the simplest sense, responsiveness is any decrease in the disease state as compared to pre-treatment, and non-responsiveness is the lack of any change in the disease state as compared to pre-treatment. Responsiveness of a human subject with a tumor can be classified based on one or more of a number of objective clinical indicia such as, but not limited to, tumor size, Clinical Benefit (CB), Progression Free Survival (PFS), Overall Survival (OS), % of maximum tumor Shrinkage (MTS), or Objective Response Rate (ORR).

“Clinical benefit” refers to having one of the following statuses—Complete Response (CR), Partial Response (PR); or Stable Disease (SD) with 6 months or more progression free survival (PFS). “Complete Response” means complete disappearance of all target lesions. “Partial Response” means at least 30% decrease in the sum of the longest diameter (LD) of target lesions, taking as reference the baseline summed LD. “Progressive Disease” (PD) means at least 20% increase in the sum of the LD of target lesions, taking as reference the smallest summed LD recorded since the treatment started, or the appearance of one or more new lesions. “Stable Disease” means neither sufficient shrinkage of the target lesions to qualify for PR nor sufficient increase to qualify for progressive disease (PD), taking as reference the smallest summed LD since the treatment started.

“Overall Survival” (OS) is defined as the time from randomization until death from any cause. “Randomization” means randomization of a patient into a test group or a control group when therapy plan for a patient is determined.

“Progression Free Survival” (PFS) refers to the time from the date of randomization to the date of first documentation of disease progression or death, whichever occurs first.

“9% of Maximum Tumor shrinkage” (MTS) means percent change of sum of diameters of target lesions, taking as reference the baseline sum diameters.

“Objective Response Rate ” (ORR) compares subjects with either Complete Response (CR) or Partial Response (PR) with subjects with either Stable Disease (SD) or Progressive Disease (PD).

### EXAMPLES

### Example 1: Selection of MVD Related Genes from the Analysis of Mouse Tumor Samples

### Materials and Methods:

scRNA-seq Using Hepa 1-6 Mouse Syngeneic Tumor Model.

Tumor tissues were resected from Hepa 1-6 mouse HCC model without treatment and dissociated into single cells by a Tumor Dissociation Kit and gentleMACS Dissociator (Miltenyi Biotec). Using the cell mixture, scRNA-seq libraries were prepared using the Chromium Controller and Chromium Single Cell 3′ library and Gel Bead Kit v3 (10× Genomics). To quantify expression levels of mRNA, sequencing of the library was performed by Illumina NovaSeq™ at GENEWIZ. Sample data processing was conducted using Cell Ranger (version 3.0.1) by Amelieff. Gene expression data of all cells were subjected to principle component analysis followed by visualization using t-SNE. 15 cell clusters were classified by their expression profile and annotated by expression of known cell marker genes. Genes that were highly expressed in endothelial cell cluster (Fold change>0 and adjusted p-value<0.05) were extracted as candidate genes for gene score. Loupe Browser (version 5.1.0. 10× Genomics) was used to find the cells expressing the genes of interest and make violin plots of six genes. “Endothelial cell” cluster was gated as Kdr positive cells, and “Others” was the cells other than endothelial cell cluster.

### RNA-seq and MVD (IHC_{CD31}) Calculation Using 12 Mouse Syngeneic Tumor Models

Twelve mouse cell lines were cultured with appropriate medium (Table 1). Cells were harvested and suspended with Hanks' Balanced Salt Solution (HBSS) and subcutaneously inoculated into right lateral side of 7-week old immune competent mice. Tumor size was measured by an electronic digital caliper (Digimatic Caliper, Mitsutoyo Corporation) and calculated with a formula; (short diameter (mm))²×(long diameter (mm))/2. Tumors around 100 mm³ were resected and soaked with RNA later. mRNA extracted from tumor samples were followed by RNA-seq using Illumina NovaSeq™ (GENEWIZ) to quantify expression levels of mRNA. Sequenced reads were subjected to quality assessment by FastQC (version 0.11.5). Illumina adaptor sequences and low quality bases were trimmed by using Trimmomatic (version 0.36). Filtered reads were mapped to the mouse genome (GRCm38/mm10 and Ensembl 88) by using STAR (v2.5.2b). Gene expression levels in transcripts per million (TPM) for all samples were estimated by using RSEM (v1.2.31). Using the same tumor samples, CD31 was stained with anti-CD31 antibody (Clone SZ31, dianova) to detect endothelial cells in IHC analysis. Microvessel density (MVD_{CD31}) score was calculated by dividing the number of CD31 positive blood vessels by tumor area using HALO (Indica Lab).

TABLE 1
Twelve mouse syngeneic tumor models

| | | Vendor/Catalog | | |
|---|---|---|---|---|
| Cell Bine | Histotype | number | Culture modium | Animal strain |
| RAG | Renal | ATCC/CCL-142 | An Eagle's minimal essential medium (E-MEM) containing | BALB/cAnNCrlCrlj, female, Charles |
| | | | 10% focal bovine serum(FBS) and penicillin/streptomycin | River Laboratories Japan Inc. |
| | | | (P/S)(100 unit/mL each) | |
| Renca | Renal | ATCC/CRL-2947 | A RPMI1640 containing 10% FBS, NEAA (0.1 mM), sodium | BALB/cAnNCrlCrlj, female, Charles |
| | | | pyruvate (1 mM), L-glutamine (2 mM) and P/S (100 unit/mL | River Laboratories Japan Inc. |
| | | | each) | |
| Hepa 1-6 | Liver | ATCC/CRL-1830 | A Dulbecco's Modified Eagle's medium (DMEM) | C57L/J, male, Breeding in-house |
| | | | containing 10% FBS and P/S (100 unit/ml, each) | |
| BNL 1MEA.7R.1 | Liver | ATCC/TIB-75 | A DMEM containing 10% FBS and P/S (100 unit/mL each) | BALB/cAnNCrlCrlj, male, Charles River |
| | | | | Laboratories Japan Inc. |
| CT26.WT | Colon | ATCC/CRL-2638 | A RPMI1640 containing 10% FBS and P/S (100 unit/mL | BALB/cAnNCrlCrlj, female, Charles |
| | | | cach) | River Laboratories Japan Inc. |
| MC38 | Colon | Kerafast/ | A DMEM containing 10% FBS and P/S (100 mit/mb cuch) | CS7BL/6NCrl, female, Charles River |
| | | ENH204-FP | | Laboratories Japan Inc. |
| 4T1 | Breast | ATCC/CRL-2539 | A RPMI1640 (ATCC-30-2001) containing 10% FBS and P/S | BALB/cAnNCrlCrlj, female, Charles |
| | | | (100 unit/mL each) | River Laboratories Japan Inc. |
| EMT6 | Mammary | ATCC/CRL-2755 | A Waymouth's (1x) MB752/1 medium containing 10% FBS, | BALB/cAnNCrlCrlj, female, Charles |
| | | | 1x GlutaMAX-1 and P/S(100 unit/mL each) | River Laboratories Japan Inc. |
| KLN205 | Lung | ATCC/CRL-1453 | An E-MEM containing 10% FBS, sodium pyruvate (1 mM) | DBA/2NCrl, female, Charles River |
| | | | and P/S (100) unit/mL each) | Laboratories Japan Inc. |
| LL/2 | Lung | ATCC/CRL-1642 | A DMEM containing 10% EBS and P/S (100 unit/mL each) | C57BL/6JCrl, female, Charles River |
| | | | | Laboratories Japan Inc. |
| B16-F10 | Melanoma | ATCC/CRL-6475 | A DMEM containing 10% FBS and P/S (100 unit/mL each) | C57BL/6NCrl, female, Charles River |
| | | | | Laboratories Japan Inc. |
| MBT2 | Bladder | JCRB/1FO50041 | An E-MEM containing 10% FBS and P/S (100 unit/mL each) | C3H/HeNCrl, female, Charles River |
| | | | | Laboratories Japan Inc. |

### Development of Mouse Gene Score

We developed a gene score by the following steps. First, we extracted 1086 genes that were highly expressed in an endothelial cell population using scRNA-seq data of the Hepa 1-6 mouse HCC model. Next, we extracted genes with TOP20 Pearson's correlation coefficient between its logarithm-transformed expression values (log₂ (TPM+1)) and MVD (IHC_{CD31}) using 12 syngeneic tumor models that were measured by RNA-seq and IHC, respectively. Of these 20 genes, we selected seven genes that had known association with vasculature from literature search and added 3 well-known endothelial cell markers to the gene list (Table 2). We further selected genes whose expression were specific to endothelial cell cluster by manually checking t-SNE plots of scRNA-seq data of Hepa 1-6 HCC model. Finally, six genes were chosen as mouse MVD gene set. The gene score of sample i was calculated as follows:

$$MVD gene {score}_{i} = \frac{1}{n} {\sum }_{j = 1}^{n} {g}_{i , j} , \qquad [ Math . 1 ]$$

where gᵢⱼ is logarithm-transformed expression level of gene j in sample i.

### Antitumor Activity of Lenvatinib on Mouse Syngeneic Tumor Models

The twelve mouse cell lines shown in Table 1 were subcutaneously inoculated into the right lateral side of 7-week-old immune competent mice. Mice were randomly grouped when the tumor size reached approximately 100 mm³. Lenvatinib (10 mg/kg once daily) was orally administered for mice of lenvatinib treatment group. The day on which treatment was started was designated as day 1. The values of antitumor activity (ΔT/C) were calculated as ΔT/ΔC×100 where ΔT and ΔC are changes in the mean tumor volumes for drug treated and untreated control groups, respectively. When the tumor volumes decreased from the initial tumor volumes, ΔT/C values were calculated as (Tₜ−T₁)/T₁×100, where Tₜ and T₁ are the mean tumor volumes of drug treated group on day t after the start of treatment and day 1, respectively. ΔT/C on day 15 was used for the analysis.

TABLE 2
Murine and human gene IDs of candidate genes for gene score

| | | NCBI gene | | | |
|---|---|---|---|---|---|
| Organism | Symbol | ID | URL | Ensembl ID | Aliases |
| Mice | Kdr | 16542 | https://www.ncbi.nlm.nih.gov/gene/16542 | ENSMUSG00000062960 | 6130401C07, Flk, Flk-, Flk-1, |
| | | | | | Flk1, Krd-1, Ly73, VEGF, |
| | | | | | VEGFR, VEGFR-2, VEGFR2, |
| | | | | | orv.sVEGFR-2 |
| | Cdh5 | 12562 | https://www.ncbi.nlm.nih.gov/gene/12562 | ENSMUSG00000031871 | 7B4, AA408225, Cd144, VE, |
| | | | | | VE-C, VE-Cad, VECD, VEcad, |
| | | | | | Vec |
| | Tek | 21687 | https://www.ncbi.nlm.nih.gov/gene/21687 | ENSMUSG00000006386 | AA517024, Cd202b, Hyk, STK1, |
| | | | | | Tie, Tie-2, Tie2, tie- |
| | Pcdh12 | 53601 | https://www.ncbi.nlm.nih.gov/gene/53601 | ENSMUSG00000024440 | Pedh14, VE-cad-2 |
| | Sparcl1 | 13602 | https://www.ncbi.nlm.nih.gov/gene/13602 | ENSMUSG00000029309 | Eem2, Sc, Sc1, he, hevin, mas, |
| | | | | | mast9 |
| | Nox4 | 50490 | https://www.ncbi.nlm.nih.gov/gene/50490 | ENSMUSG00000030562 | AI648021 |
| | Notch4 | 18132 | https://www.ncbi.nlm.nih.gov/gene/18132 | ENSMUSG00000015468 | In, Int, Int-3, Int3, N, N4 |
| | Eng | 13805 | https://www.ncbi.nlm.nih.gov/gene/13805 | ENSMUSG00000026814 | AI528660. AI662476, CD105, En, |
| | | | | | Endo, S-endoglin |
| | Plxnd1 | 67784 | https://www.ncbi.nlm.nih.gov/gene/67784 | ENSMUSG00000030123 | 6230425C21Rik, b2b1863C, |
| | | | | | b2b1863Clo,b2b553C, b2b553Clp |
| | Arhgap29 | 214137 | https://www.ncbi.nlm.nih.gov/gene/214137 | ENSMUSG000000039831 | 6720461J18Rik, A830014I19, |
| | | | | | AU040217, B130017101Rik, |
| | | | | | C76601, Pa, Parg1 |
| Human | KDR | 3791 | https://www.ncbi.nlm.nih.gov/gene/3791 | ENSG00000128052 | VEGFR2, FLK1, CD309 |

TABLE 3

| | | | | |
|---|---|---|---|---|
| CDH5 | 1003 | https://www.ncbi.nlm.nih.gov/gene/1003 | ENSG00000179776 | Cadherin 5, VE-Cadherin, CD144 |
| TEK | 7010 | https://www.ncbi.nlm.nih.gov/gene/7010 | ENSG00000120156 | TIE2, VMCM1, Angiopoetin-1 |
| | | | | Receptor |
| PCDH12 | 51294 | https://www.ncbi.nlm.nih.gov/gene/51294 | ENS000000113535 | VE-Cadherin-2, Protocadherin 12 |
| SPARCL1 | 8404 | https://www.ncbi.nlm.nih.gov/gene/8404 | ENS000000152583 | MAST9, Hevin, PlG33, SC1 |
| NOX4 | 50507 | https://www.ncbi.nlm.nih.gov/gene/50507 | ENSG00000086991 | KOX,KOX-1, RENOX |
| NOTCH4 | 4855 | https://www.ncbi.nlm.nih.gov/gene/4855 | ENSG00000204301 | INT3 |
| ENG | 2022 | https://www.ncbi.nlm.nih.gov/gene/2022 | ENSG00000106991 | Endoglin, CD105, ORW1, HHT1 |
| PLAND1 | 23129 | https://www.ncbi.nlm.nih.gov/gene/23129 | ENSG00000004399 | PLEXD1 |
| ARHGAP29 | 9411 | https://www.ncbi.nlm.nih.gov/gene/9411 | ENSG00000137962 | PARG1 |

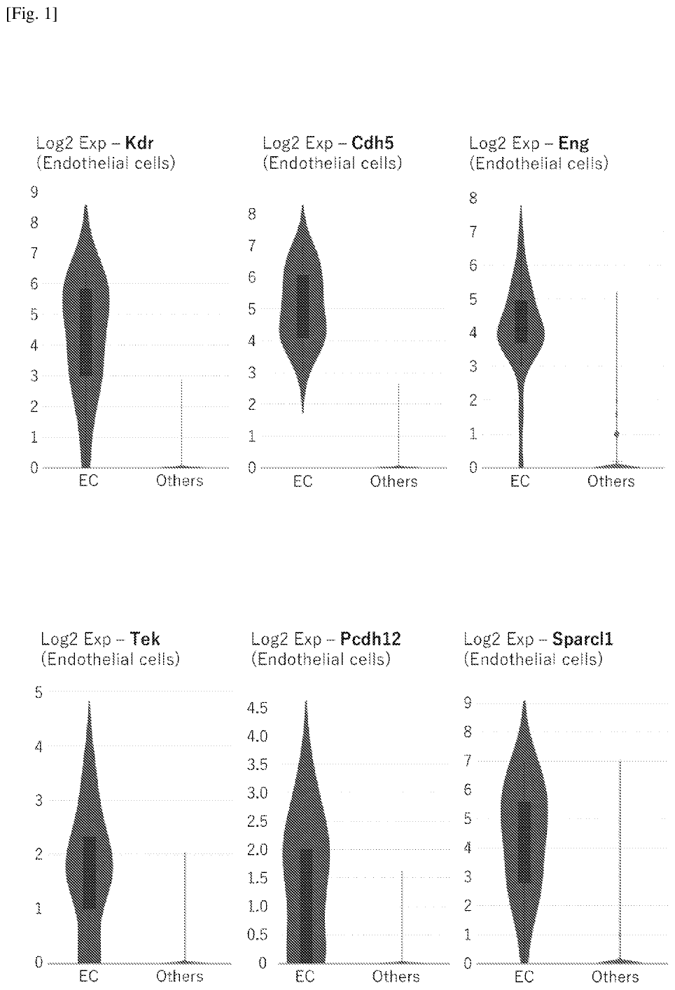

Results: MVD related genes were selected by filtering endothelial cell specific genes using scRNA-seq data of the Hepa 1-6 mouse liver tumor model and selected genes whose expression levels measured by RNA-seq were correlated with MVD (IHC_{CD31}) in pre-treatment tumor samples across 12 mouse syngeneic tumor models (n=3 for each tumor model except for MC38 tumor where n=2). MVD (IHC_{CD31}) correlated genes with known association to angiogenesis and well-known endothelial cell markers were chosen for a candidate gene list (Table 2). Kdr, Cdh5, Eng, Tek, Pcdh12, and Sparcl1 were further selected as MVD related genes according to their specificity in expression on endothelial cells. FIG. 1 shows expression of each gene on scRNA-seq data. All genes were specifically expressed on endothelial cell cluster compared to other clusters.

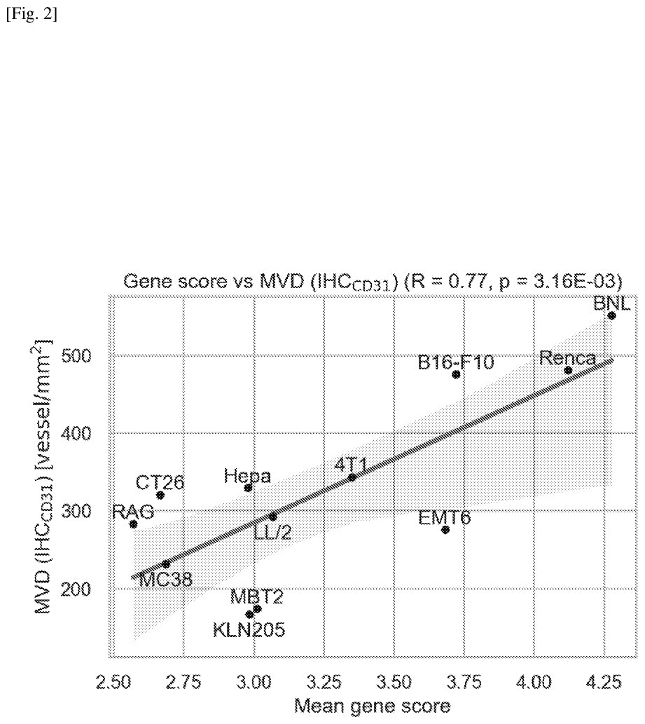

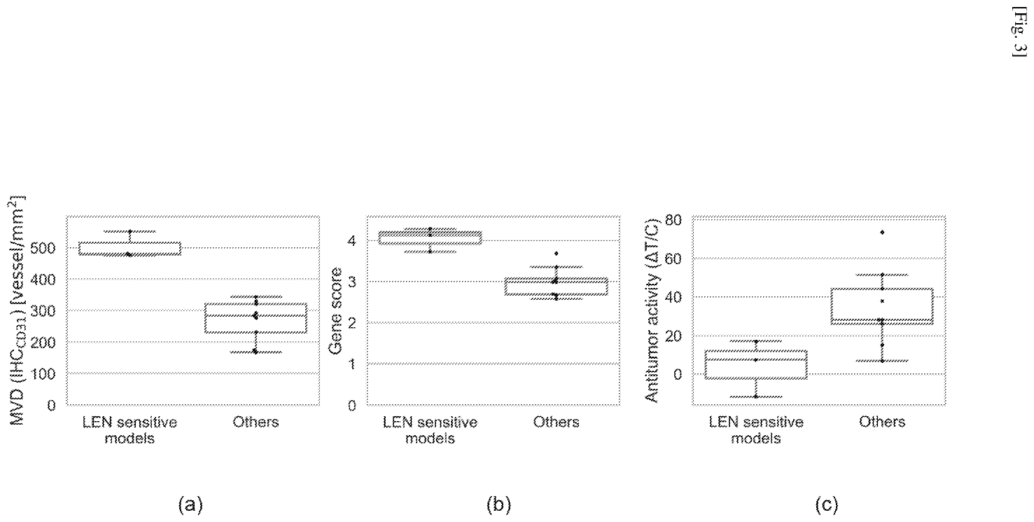

To evaluate the gene score from gene expression levels of these six genes, we defined the gene score as the average of logarithm-transformed mRNA expression levels of genes and compared it with MVD (IHC_{CD31}). MVD (IHC_{CD31}) and the gene score computed from gene expression levels of six genes showed significant correlation (R=0.77, p<0.01) (FIG. 2). Lenvatinib sensitive models (i.e., BNL, Renca, and B16-F10) showed higher mean MVD (IHC_{CD31}) (p<0.001, t-test) (FIG. 3a), mean gene score (p<0.01, t-test) (FIG. 3b), and more antitumor activity by lenvatinib (p<0.05, t-test) (FIG. 3c) compared to other nine tumor models.

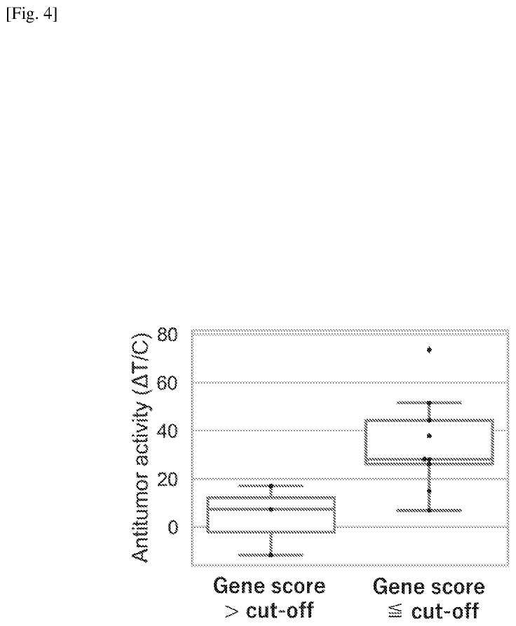

To evaluate if a pre-established cut-off value of the gene score can be determined, ΔT/C data of 12 tumor models used in FIG. 3c were sorted by order of gene score. When a 75^{th} percentile cut-off (3.69) was used for the dichotomized analysis, ΔT/C data were divided into two groups with low ΔT/C and high ΔT/C (p<0.05, t-test) (FIG. 4). This suggests that a pre-established cut-off value of the gene score can be used to predict a tumor response to lenvatinib.

### Example 2: Confirmation of Correlation of MVD (IHC_{CD31}) and Gene Score on Human Tumor Samples

As a next step, it was investigated whether a gene score that was defined through the analysis of mouse tumor samples correlates with MVD evaluated by IHC on human tumor samples.

### Materials and Methods:

### RNA-seq and MVD (IHC_{CD31}) Calculation Using Human Tumor Tissue Samples

Twelve cancer types of human tumor tissue formalin-fixed paraffin-embedded (FFPE) samples were obtained from patients who provided informed consent and acquired through the Eisai Biobank in accordance with an approved protocol. Using the human FFPE tumor block samples, a 10 microM slice was cut and bedded on a glass slide, and then tumor tissues were scratched and RNA was extracted. Using the isolated RNA samples, RNA-seq was performed by NextSeq 550 sequence system (Illumina) to quantify expression levels of mRNA. Sequenced reads were subjected to quality assessment by FastQC (version 0.11.5). Illumina adaptor sequences and low quality bases were trimmed by using Trimmomatic (version 0.36). Filtered reads were mapped to the human genome (GRCh38 and Ensembl 88) by using STAR (v2.5.2b). Gene expression levels in transcripts per million (TPM) for all samples were estimated by using RSEM (v1.2.31). To detect endothelial cells, CD31 was stained with anti-CD31 antibody (Clone JC70, Roche) in tumor tissues by VENTANA Discovery XT (Roche). To avoid a noise of CD31 positive lymphocytes, tumor tissue samples infiltrated with CD31 positive lymphocytes were eliminated by judging the cell shape for further analysis. The selected twenty-four samples from ten cancer types of human tissues were analyzed as the same method described in IHC analysis using mouse tumor samples. MVD (IHC_{CD31}) score was calculated using HALO.

### Correlation Analysis of MVD (IHC_{CD31}) and Gene Score

Correlation coefficient of MVD (IHC_{CD31}) and gene expression data in 24 human tumor samples were assessed by Pearson's correlation coefficient. Correlation coefficient between MVD (IHC_{CD31}) and gene score and correlation coefficient between MVD (IHC_{CD31}) and other possible combinations of six genes (at least two genes) were tested, respectively. Gene scores were calculated as the average of logarithm-transformed expression of genes. No multiple test correction were applied in this analysis.

### Results:

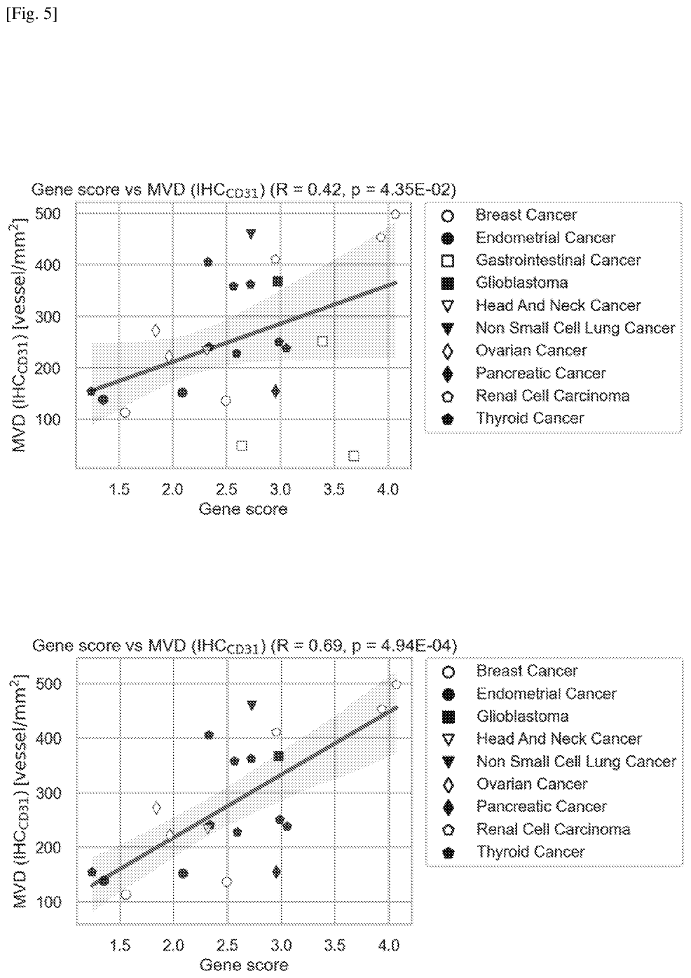

Twenty-four human tumor samples across 10 tumor types for which both IHC and RNA-seq data were available were used for the analysis (Table 3). The gene scores of these samples were calculated as the average of logarithm-transformed mRNA expression levels by using mRNA expression levels of human orthologues of six MVD related genes. FIG. 5 shows correlation of MVD (IHC_{CD31}) and gene score across 24 samples and 21 samples in which three gastrointestinal cancer samples were removed from 24 samples. MVD (IHC_{CD31}) and gene score of 24 human samples showed significant correlation (R=0.42, p=0.043). Three gastrointestinal cancer samples showed high gene score but low MVD (IHC_{CD31}). Without three gastrointestinal cancer samples, MVD (IHC_{CD31}) and gene score across 21 samples showed stronger correlation (R=0.69, p<0.001).

TABLE 4
Ten human tumor types selected for gene score validation

| Tumor Type | Number of Samples |
|---|---|
| Thyroid Cancer | 8 |
| Breast Cancer | 2 |
| Endometrial Cancer | 2 |
| Gastrointestinal Cancer | 3 |
| Glioblastoma | 1 |
| Head and Neck Cancer | 1 |
| Non small Cell Lung Cancer (NSCLC) | |
| Ovarian Cancer | 2 |
| Pancreatic Cancer | 1 |
| Renal Cell Carcinoma (RCC) | 3 |

To investigate if other combinations of genes using these six genes also show correlation to MVD (IHC_{CD31}), we calculated gene score and assessed correlation with MVD (IHC_{CD31}) for each combination of genes using two to five genes. About half of all gene combinations, gene score calculated as the average of logarithm-transformed mRNA expression levels of genes showed significant correlation to MVD (IHC_{CD31}) in 24 human tumor samples across 10 tumor types (Table 4). Without gastrointestinal cancer samples, gene score of all combinations in 21 human tumor samples showed significant correlation to MVD (IHC_{CD31}) (Table 5).

TABLE 5
Correlation of MVD (IHC_{CD31}) and gene score computed from other
combinations of MVD-related genes in 10 cancer types

| index | Gene combination | pearson r | pearson p | spearman r | spearman p |
|---|---|---|---|---|---|
| 1 | KDR_CDH5 | 0.79 | 3.56E−06 | 0.79 | 4.88E−06 |
| 2 | KDR_CDH5_TEK | 0.77 | 1.33E−05 | 0.72 | 6.66E−05 |
| 3 | KDR_TEK | 0.76 | 1.44E−05 | 0.68 | 2.32E−04 |
| 4 | ENG_KDR_CDH5_TEK | 0.66 | 4.14E−04 | 0.63 | 9.59E−04 |
| 5 | ENG_KDR_CDH5 | 0.66 | 5.07E−04 | 0.63 | 1.02E−03 |
| 6 | ENG_KDR_TEK | 0.65 | 6.15E−04 | 0.64 | 6.78E−04 |
| 7 | CDH5_TEK | 0.65 | 6.29E−04 | 0.62 | 1.16E−03 |
| 8 | PCDH12_KDR_CDH5_TEK | 0.64 | 7.36E−04 | 0.52 | 3.67E−03 |
| 9 | ENG_KDR | 0.64 | 8.32E−04 | 0.59 | 2.34E−03 |
| 10 | PCDH12_KDR_TEK | 0.62 | 1.32E−03 | 0.53 | 7.32E−03 |
| 11 | PCDH12_KDR_CDH5 | 0.62 | 1.35E−03 | 0.52 | 9.62E−03 |
| 12 | PCDH12_KDR | 057 | 3.47E−03 | 0.34 | 6.77E−03 |
| 13 | ENG_PCDH12_KDR_CDH5_TEK | 0.57 | 3.70E−03 | 0.48 | 1.71E−02 |
| 14 | ENG_PCDH12_KDR_CDH5 | 0.54 | 6.62E−03 | 0.49 | 1.46E−02 |
| 15 | ENG_PCDH12_KDR_TEK | 0.54 | 6.76E−03 | 0.49 | 1.60E−02 |
| 16 | KDR_SPARCL1_CDH5_TEK | 0.52 | 9.38E−03 | 0.52 | 8.79E−03 |
| 17 | ENG_CDH5_TEK | 0.52 | 1.00E−02 | 0.47 | 2.06E−02 |
| 18 | ENG_PCDH12_KDR | 0.49 | 1.46E−02 | 0.49 | 1.54E−02 |
| 19 | ENG_KDR_SPARCL1_CDH5_TEK | 8.47 | 1.97E−02 | 0.44 | 3.26E−02 |
| 20 | KDR_SPARCL1_CDH5 | 0.46 | 2.39E−02 | 0.47 | 2.14E−03 |
| 21 | PCDH12_CDH5_TEK | 0.36 | 2.52E−02 | 0.40 | 5.45E−02 |
| 22 | ENG_CDH5 | 0.45 | 2.72E−02 | 0.45 | 2.85E−02 |
| 23 | KDR_SPARCL1_TEK | 0.45 | 2.74E−02 | 0.45 | 2.89E−02 |
| 24 | PCDH12_KDR_SPARCL1_CDH5_TEK | 0.44 | 3.04E−02 | 0.48 | 1.76E−02 |
| 25 | ENG_KDR_SPARCL1_CDH5 | 0.42 | 3.92E−02 | 0.43 | 3.54E−02 |
| 26 | ENG_KDR_SPARCL1_TEK | 0.42 | 4.36E−02 | 0.37 | 7.33E−02 |
| 27 | ENG_TEK | 0.43 | 4.42E−02 | 0.36 | 8.24E−02 |
| 28 | ENG_PCDH12_CDH5_TEK | 0.41 | 4.81E−02 | 0.40 | 5.17E−02 |
| 29 | PCDH12_KDR_SPARCL1_CDH5 | 0.38 | 6.39E−02 | 0.40 | 5.57E−02 |
| 30 | PCDH12_KDR_SPARCL1_TEK | 8.38 | 7.08E−02 | 0.39 | 5.62E−02 |
| 31 | ENG_PCDH12_KDR_SPARCL1_CDH5 | 0.37 | 7.72E−02 | 0.39 | 5.86E−02 |
| 32 | ENG_PCDH12_KDR_SPARCL1_TEK | 0.36 | 8.45E−02 | 0.38 | 6.70E−02 |
| 33 | KDR_SPARCL1 | 0.35 | 8.96E−02 | 0.36 | 8.40E−02 |
| 34 | PCDH12_CDH5 | 0.35 | 9.44E−02 | 0.33 | 1.01E−01 |
| 35 | ENG_KDR_SPARCL1 | 0.35 | 9.87E−02 | 0.34 | 1.01E−01 |
| 36 | ENG_PCDH12_CDH5 | 0.33 | 1.14E−01 | 0.33 | 1.17E−01 |
| 37 | PCDH12_TEK | 0.31 | 1.44E−01 | 0.35 | 9.85E−02 |
| 38 | ENG_PCDH12_TEK | 0.38 | 1.46E−01 | 0.28 | 1.88E−01 |
| 39 | ENG_PCDH12_KDR_SPARCL1 | 0.30 | 1.60E−01 | 0.34 | 1.05E−01 |
| 40 | PCDH12_ KDR_SPARCL1 | 0.29 | 1.65E−01 | 0.33 | 1.11E−01 |
| 41 | ENG_SPARCL1_CDH5_TEK | 0.28 | 1.88E−01 | 0.36 | 8.24E−02 |
| 42 | SPARCL1_CDH5_TEK | 0.27 | 2.04E−01 | 0.37 | 7.62E−02 |

TABLE 6

| | | | | | |
|---|---|---|---|---|---|
| 43 | ENG_PCDH12_SPARCL1_CDH5_TEK | 0.24 | 2.49E−01 | 0.27 | 1.92E−01 |
| 44 | PCDH12_SPARCL1_CDH5_TEK | 0.23 | 2.81E−01 | 0.28 | 1.87E−01 |
| 45 | ENG_SPARCL1_CDH5 | 0.19 | 3.79E−01 | 0.32 | 1.27E−01 |
| 46 | ENG_PCDH12 | 0.18 | 4.02E−01 | 0.25 | 2.48E−01 |
| 47 | ENG_PCDH12_SPARCL1_CDH5 | 0.17 | 4.26E−01 | 0.24 | 2.60E−01 |
| 48 | ENG_SPARCL1_TEK | 0.15 | 4.88E−01 | 0.23 | 2.77E−01 |
| 49 | ENG_PCDH12_SPARCL1_TEK | 0.14 | 5.13E−01 | 0.22 | 2.94E−01 |
| 50 | SPARCL1_CDH5 | 0.14 | 5.18E−01 | 0.28 | 1.92E−01 |
| 51 | PCDH12_SPARCL1_CDH5 | 0.13 | 5.41E−01 | 0.23 | 2.88E−01 |
| 52 | PCDH12_SPARCL1_TEK | 0.09 | 6.91E−01 | 0.19 | 3.75E−01 |
| 53 | SPARCL1_TEK | 0.07 | 7.40E−01 | 0.15 | 4.83E−01 |
| 54 | ENG_PCDH12_SPARCL1 | 0.05 | 8.32E−01 | 0.17 | 4.24E−01 |
| 55 | ENG_SPARCL1 | 0.02 | 9.13E−01 | 0.16 | 4.65E−01 |
| 56 | PCDH12_SPARCL1 | −0.04 | 8.46E−01 | 0.33 | 5.46E−01 |

TABLE 7
Correltion of MVD (IHC_{CD31}) and gene score computed from other
combinations of MVD-related genes in 9 cancer types (cancer types of Table 4 other
than gastrointestinal cancer)

| index | Gene combination | pearson r | pearson p | spearman r | spearman p |
|---|---|---|---|---|---|
| 1 | KDR_CDH5 | 0.81 | 9.90E−06 | 0.79 | 2.19E−05 |
| 2 | PCDH12_KDR_CDH5 | 0.76 | 6.03E−05 | 0.67 | 8.88E−04 |
| 3 | KDR_CDH5_TEK | 0.76 | 6.70E−05 | 0.71 | 3.08E−04 |
| 4 | ENG_KDR_CDH5 | 0.75 | 9.59E−05 | 0.70 | 4.26E−04 |
| 5 | PCDH12_KDR | 0.74 | 1.13E−04 | 0.68 | 6.42E−04 |
| 6 | PCDH12_KDR_CDH5_TEK | 0.74 | 1.37E−04 | 0.71 | 3.20E−04 |
| 7 | KDR_TEK | 0.74 | 1.40E−04 | 0.67 | 910E−04 |
| 8 | ENG_KDR | 0.73 | 1.86E−04 | 0.68 | 7.57E−04 |
| 9 | ENG_PCDH12_KDR_CDH5 | 0.73 | 1.86E−04 | 0.65 | 1.58E−03 |
| 10 | PCDH12_KDR_TEK | 0.72 | 2.34E−04 | 0.65 | 1.28E−03 |
| 11 | ENG_KDR_CDH5_TEK | 0.72 | 2.43E−04 | 0.68 | 6.64E−04 |
| 12 | KDR_SPARCL1_CDH5 | 0.72 | 2.52E−04 | 0.72 | 2.11E−04 |
| 13 | ENG_KDR_SPARCL1_CDH5 | 0.71 | 3.01E−04 | 0.68 | 7.57E−04 |
| 14 | KDR_SPARCL1_CDH5_TEK | 0.71 | 3.03E−04 | 0.71 | 3.56E−04 |
| 15 | ENG_PCDH12_KDR_CDH5_TEK | 0.71 | 3.17E−04 | 0.61 | 3.21E−03 |
| 16 | ENG_PCDH12_KDR | 0.71 | 3.22E−04 | 0.63 | 2.15E−03 |
| 17 | PCDH12_KDR_SPARCL1_CDH5 | 0.70 | 3.68E−04 | 0.64 | 1.62E−03 |
| 18 | PCDH12_KDR_SPARCL1_CDH5_TEK | 0.70 | 3.96E−04 | 0.68 | 6.42E−04 |
| 19 | ENG_KDR_SPARCL1_CDH5_TEK | 0.70 | 4.08E−04 | 0.66 | 1.01E−03 |
| 20 | ENG_PCDH12_KDR_SPARCL1_CDH5 | 0.70 | 4.15E−04 | 0.64 | 1.77E−03 |
| 21 | ENG_KDR_TEK | 0.69 | 4.79E−04 | 0.68 | 7.57E−04 |
| 22 | ENG_PCDH12_KDR_TEK | 0.69 | 5.31E−04 | 0.63 | 2.09E−03 |
| 23 | ENG_KDR_SPARCL1 | 0.68 | 7.32E−04 | 0.63 | 2.15E−03 |
| 24 | ENG_KDR_SPARCL1_TEK | 0.67 | 8.26E−04 | 0.62 | 2.60E−03 |
| 25 | ENG_PCDH12_KDR_SPARCL1 | 0.67 | 8.26E−04 | 0.62 | 2.82E−03 |
| 26 | PCDH12_KDR_SPARCL1_TEK | 0.67 | 8.65E−04 | 0.64 | 1.93E−03 |
| 27 | ENG_PCDH12_KDR_SPARCL1_TEK | 0.67 | 8.69E−04 | 0.63 | 2.40E−03 |
| 28 | KDR_SPARCL1_TEK | 0.67 | 8.87E−04 | 0.66 | 1.25E−03 |
| 29 | PCDH12_CDH5 | 0.67 | 9.68E−04 | 0.48 | 2.94E−02 |
| 30 | ENG_CDH5 | 0.66 | 1.02E−03 | 0.62 | 2.89E−03 |
| 31 | PCDH12_KDR_SPARCL1 | 0.66 | 1.07E−03 | 0.61 | 3.21E−03 |
| 32 | KDR_SPARCL1 | 0.66 | 1.21E−03 | 0.63 | 2.40E−03 |
| 33 | CDH5_TEK | 0.66 | 1.28E−03 | 0.62 | 2.82E−03 |
| 34 | PCDH12_CDH5_TEK | 0.65 | 1.37E−03 | 0.52 | 1.49E−02 |
| 35 | ENG_PCDH12_CDH5 | 0.65 | 1.37E−03 | 0.48 | 2.65E−02 |
| 36 | ENG_PCDH12_CDH5_TEK | 0.64 | 1.86E−03 | 0.54 | 1.24E−02 |
| 37 | ENG_CDH5_TEK | 0.63 | 2.11E−03 | 0.59 | 5.15E−03 |
| 38 | ENG_PCDH12_SPARCL1_CDH5_TEK | 0.62 | 2.53E−03 | 0.55 | 9.48E−03 |
| 39 | ENG_PCDH12_SPARCL1_CDH5 | 0.62 | 2.76E−03 | 0.54 | 1.15E−02 |
| 40 | ENG_SPARCL1_CDH5_TEK | 0.62 | 2.88E−03 | 0.65 | 1.36E−03 |

TABLE 8

| | | | | | |
|---|---|---|---|---|---|
| 41 | ENG_SPARCL1_CDH5 | 0.61 | 3.09E−03 | 0.65 | 1.32E−03 |
| 42 | PCDH12_SPARCL1_CDH5_TEK | 0.61 | 3.44E−03 | 0.55 | 9.08E−03 |
| 43 | SPARCL1 CDH5_TEK | 0.59 | 4.80E−03 | 0.63 | 2.34E−03 |
| 44 | PCDH12_SPARCL1_CDH5 | 0.59 | 5.20E−03 | 0.52 | 1.40E−02 |
| 45 | ENG_PCDH12 | 0.58 | 5.34E−03 | 0.39 | 8.42E−02 |
| 46 | ENG_PCDH12_TEK | 0.58 | 5.43E−03 | 0.47 | 3.26E−02 |
| 47 | ENG_PCDH12_SPARCL1_TEK | 0.57 | 6.71E−03 | 0.52 | 1.64E−02 |
| 48 | PCDH12_TEK | 0.57 | 7.21E−03 | 0.50 | 2.14E−02 |
| 49 | SPARCL1_CDH5 | 0.56 | 8.89E−03 | 0.59 | 4.68E−03 |
| 50 | ENG_PCDH12_SPARCL1 | 0.55 | 9.64E−03 | 0.46 | 3.43E−02 |
| 51 | ENG_TEK | 0.54 | 1.07E−02 | 0.49 | 2.43E−02 |
| 52 | ENG_SPARCL1_TEK | 0.54 | 1.08E−02 | 0.55 | 9.69E−03 |
| 53 | PCDH12_SPARCL1_TEK | 0.52 | 1.51E−02 | 0.46 | 3.48E−02 |
| 54 | ENG_SPARCL1 | 0.51 | 1.83E−02 | 0.50 | 2.18E−02 |
| 55 | PCDH12_SPARCL1 | 0.46 | 3.74E−02 | 0.42 | 5.75E−02 |
| 56 | SPARCL1_TEK | 0.45 | 4.20E−02 | 0.47 | 3.15E−02 |

### Example 3: Confirmation of Correlation of MVD (IHC_{CD34}) and Gene Score on Human Tumor Samples

### Materials and Methods:

### RNA-seq and MVD (IHC_{CD34}) Calculation Using Human Tumor Tissue Samples

MVD (IHC_{CD34}) of human tumor samples was assessed by IHC analysis of CD34, which is a well-known endothelial marker in human tissues. Formalin-fixed paraffin-embedded (FFPE) human tumor samples were stained with anti-CD34 antibody (clone QBEnd/10, Leica Biosystems) by BOND-RX (Leica Biosystems) using the same human tumor tissue samples as Example 2. MVD (IHC_{CD34}) score was calculated by dividing the number of CD34 positive blood vessels by tumor area using HALO (Indica Lab). Three gastrointestinal cancer samples were excluded from the analysis because not only microvessels but also whole tissues were stained by the anti-CD34 antibody. One glioblastoma sample and two melanoma samples were excluded from the analysis because these samples did not pass sample QC on RNA-seq data.

### Correlation Analysis of MVD (IHC_{CD34}) and Gene Score

Correlation of MVD (IHC_{CD34}) computed from IHC analysis of CD34 and gene score was assessed by Pearson's correlation coefficient. Gene score was calculated as the average of logarithm-transformed expression values (log₂(TPM+1)) of six genes.

### Results:

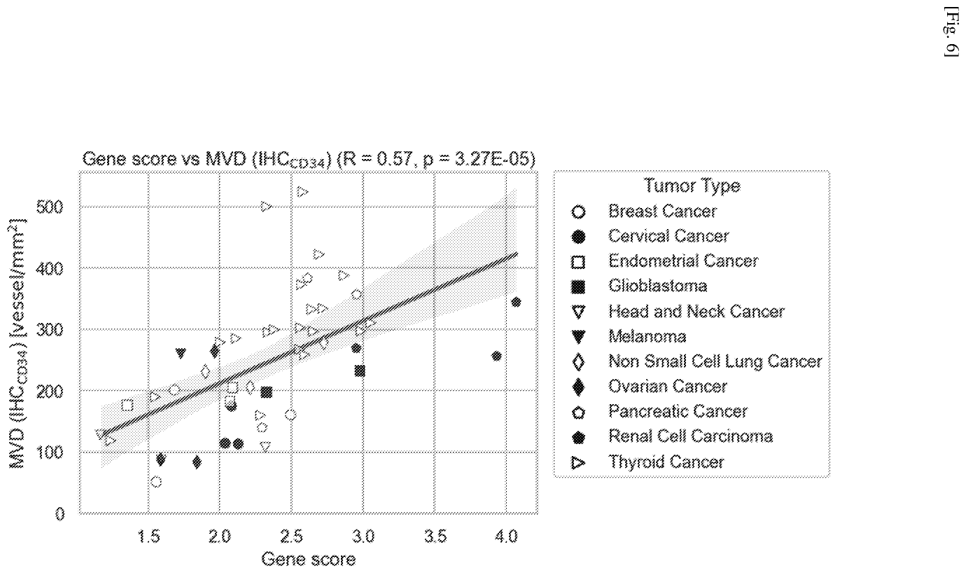

Forty-seven human tumor samples across 11 tumor types for which both IHC data of CD34 and RNA-seq data were available were used for the analysis (Table 6). FIG. 6 shows correlation of MVD (IHC_{CD34}) and gene score across 47 samples (R=0.57, p<0.05). These results indicate that gene score correlates with MVD (IHC) when MVD (IHC) is assessed by different endothelial markers (i.e., CD31 or CD34).

To investigate if other combinations of genes using these six genes also show correlation to MVD (IHC_{CD34}), we calculated gene score and assessed correlation with MVD (IHC_{CD34}) for each combination of genes using two to five genes (Table 7). Gene score calculated as the average of logarithm-transformed mRNA expression levels of genes showed significant correlation to MVD (IHC_{CD34}).

These results indicate that gene score calculated from the expression levels of at least two of the six genes correlates with MVD (IHC) even when MVD (IHC) is assessed by different endothelial cell markers (i.e., CD31 or CD34).

TABLE 9
Eleven human tumor types selected for gene score validation

| Tumor Type | Number of Samples |
|---|---|
| Thyroid Cancer | 20 |
| Breast Cancer | 3 |
| Cervical Cancer | 3 |
| Endometrial Cancer | 3 |
| Glioblastoma | 2 |
| Head and Neck Cancer | 3 |
| Melanoma | 1 |
| Non-small Cell Lung Cancer | 3 |
| Ovarian Cancer | 3 |
| Pancreatic Cancer | 3 |
| Renal Cell Carcinoma | 3 |

TABLE 10
Correltion of MVD (IHC_{CD54}) and gene score computed from other
combinations of MVD-related genes in 11 cancer types

| index | Gene combination | pearson r | pearson p | spearman r | spearman p |
|---|---|---|---|---|---|
| 1 | CDH5_TEK | 0.62 | 3.62E−06 | 0.74 | 2.50E−09 |
| 2 | KDR_CDH5_TEK | 0.62 | 4.17E−06 | 0.73 | 7.61E−09 |
| 3 | ENG_KDR_CDH5_TEK | 0.60 | 8.23E−06 | 0.73 | 7.55E−09 |
| 4 | KDR_TEK | 0.60 | 8.45E−06 | 0.74 | 3.30E−09 |
| 5 | KDR_CDH5 | 0.59 | 1.26E−05 | 0.71 | 2.36E−08 |
| 6 | ENG CDH5_TEK | 0.59 | 1.27E−05 | 0.71 | 3.09E−08 |
| 7 | PCDH12_KDR_CDH5_TEK | 0.59 | 1.33E−05 | 0.71 | 1.68E−08 |
| 8 | ENG_PCDH12_KDR_CDH5_TEK | 0.59 | 1.57E−05 | 0.73 | 4.41E−09 |
| 9 | ENG_KDR_TEK | 0.58 | 1.57E−05 | 0.70 | 3.28E−08 |
| 10 | ENG_KDR_CDH5 | 0.58 | 1.93E−05 | 0.71 | 1.93E−08 |
| 11 | PCDH12_CDH5_TEK | 0.58 | 2.29E−05 | 0.69 | 1.01E−07 |
| 12 | ENG_PCDH12_CDH5_TEK | 0.57 | 2.52E−05 | 0.73 | 1.66E−08 |
| 13 | ENG_KDR_SPCDH12_CDH5_TEK | 0.57 | 2.69E−05 | 0.69 | 7.23E−08 |
| 14 | ENG_PCDH12_KDR_TEK | 0.57 | 2.78E−05 | 0.73 | 6.35E−09 |
| 15 | PCDH12_KDR_TEK | 0.57 | 2.85E−05 | 0.70 | 4.57E−08 |
| 16 | KDR_SPARCL1_CDH5_TEK | 0.57 | 3.29E−05 | 0.68 | 1.24E−07 |
| 17 | ENG_PCDH12_KDR_CDH5 | 0.56 | 3.80E−05 | 0.70 | 4.57E−08 |
| 18 | PCDH12_KDR_SPARCL1_CDH5_TEK | 0.56 | 4.10E−05 | 0.67 | 2.18E−07 |
| 19 | ENG_CDH5 | 0.56 | 4.18E−05 | 0.65 | 7.33E−07 |
| 20 | PCDH12_KDR_CDH5 | 0.56 | 4.50E−05 | 0.66 | 4.84E−07 |
| 21 | ENG_TEK | 0.55 | 5.90E−05 | 0.66 | 4.21E−07 |
| 22 | ENG_KDR | 0.55 | 5.96E−05 | 0.68 | 1.56E−07 |
| 23 | ENG_KDR_SPARCL1_CDH5 | 0.55 | 6.79E−05 | 0.66 | 4.32E−07 |
| 24 | ENG_PCDH12_TEK | 0.55 | 7.11E−05 | 0.69 | 9.63E−08 |
| 25 | ENG_CDH12_KDR_SPARCL1_TEK | 0.55 | 7.45E−05 | 0.66 | 3.51E−07 |
| 26 | ENG_PCDH12_KDR_SPARCL1_CDH5 | 0.54 | 7.64E−05 | 0.67 | 1.96E−07 |
| 27 | ENG_KDR_SPARCL1_TEK | 0.54 | 7.87E−05 | 0.65 | 6.90E−07 |
| 28 | ENG_PCDH12_SPARCL1_CDH5_TEK | 0.54 | 8.32E−05 | 0.65 | 6.30E−07 |
| 29 | ENG_PCDH12_CDH5 | 0.34 | 8.97E−05 | 0.67 | 3.03E−07 |
| 30 | ENG_SPARCL1_CDH5_TEK | 0.54 | 9.41E−05 | 0.64 | 1.38E−06 |
| 31 | ENG_PCDH12_KDR | 0.54 | 9.75E−05 | 0.69 | 6.93E−08 |
| 32 | KDR_SPARCL1_CDH5 | 0.53 | 1.20E−04 | 0.64 | 1.36E−06 |
| 33 | PCDH12_KDR_SPARCL1_CDH5 | 0.53 | 1.26E−04 | 0.63 | 2.01E−06 |
| 34 | PCDH12_TEK | 0.53 | 1.28E−04 | 0.68 | 1.77E−07 |
| 35 | PCDH12_KDR_SSPARCL1_TEK | 0.53 | 1.31E−04 | 0.62 | 2.93E−06 |
| 36 | PCDH12_SPARCL1_CDH5_TEK | 0.52 | 1.61E−04 | 0.61 | 4.74E−06 |
| 37 | KDR_SPARCL1_TEK | 0.52 | 1.63E−04 | 0.60 | 9.35E−06 |
| 38 | PCDH12_CDH5 | 0.52 | 1.75E−04 | 0.61 | 5.35E−06 |
| 39 | PCDH12_KDR | 0.52 | 2.00E−04 | 0.65 | 6.78E−07 |
| 40 | SPARCL1_CDH5_TEK | 0.51 | 2.30E−04 | 0.61 | 6.19E−06 |
| 41 | ENG_PCDH12_KDR_SPARCL1 | 0.51 | 2.33E−04 | 0.61 | 4.82E−06 |
| 42 | ENG_PCDH12_SPARCL1_CDH5 | 0.51 | 2.77E−04 | 0.63 | 2.50E−06 |
| 43 | ENG_KDR_SPARCL1 | 0.50 | 2.99E−04 | 0.60 | 8.93E−06 |
| 44 | ENG_PCDH12_SPARCL1_TEK | 0.50 | 3.43E−04 | 0.59 | 1.14E−05 |
| 45 | ENG_SPARCL1_CDH5 | 0.50 | 3.93E−04 | 0.60 | 9.68E−06 |
| 46 | ENG_PCDH12 | 0.48 | 5.96E−04 | 0.61 | 6.59E−06 |
| 47 | PCDH12_KDR_SPARCL1 | 0.48 | 6.41E−04 | 0.58 | 1.57E−05 |

TABLE 11

| | | | | | |
|---|---|---|---|---|---|
| 48 | ENG_SPARCL1_TEK | 0.48 | 7.13E−04 | 0.54 | 8.91E−05 |
| 49 | PCDH12_SPARCL1_CDH5 | 0.47 | 8.97E−04 | 0.58 | 1.94E−05 |
| 50 | KDR_SPARCL1 | 0.46 | 1.17E−03 | 0.56 | 5.10E−05 |
| 51 | PCDH12_SPARCL1 TEK | 0.45 | 1.40E−03 | 0.56 | 4.43E−05 |
| 52 | ENG_PCDH12_SPARCL1 | 0.44 | 1.92E−03 | 0.54 | 8.19E−05 |
| 53 | SPARCL1_CDH5 | 0.44 | 2.04E−03 | 0.53 | 1.28E−04 |
| 54 | SPARCL1_TEK | 0.40 | 5.65E−03 | 0.48 | 7.02E−04 |
| 55 | ENG_SPARCL1 | 0.39 | 6.17E−03 | 0.44 | 2.08E−03 |
| 56 | PCDH12_SPARCL1 | 0.35 | 1.59E−02 | 0.46 | 1.11E−03 |

### Example 4: Changes in MVD Gene Score of 12 Mouse Syngeneic Tumor Models with or Without Angiogenesis Inhibitor Treatment

Aflibercept is an inhibitor of VEGF which binds to circulating VEGF and acts as VEGF-trap, leading to inhibiting tumor angiogenesis. (Holash et. al, Proc Natl Acad Sci USA 99(17):11393-8, 2002). Lenvatinib-treated tumors and aflibercept-treated tumors were subjected to RNA-seq analysis to investigate changes in gene score induced by the angiogenesis inhibitors.

### Materials and Methods:

The twelve mouse cell lines shown in Table 1 were subcutaneously inoculated into right lateral side of 7-week-old immune competent mice. Mice were randomly grouped into non-treatment and lenvatinib treatment groups when the tumor size reached approximately 100 mm³ (n=3 for each group). Lenvatinib (10 mg/kg once daily) was orally administered for mice of lenvatinib treatment group and aflibercept (5 mg/kg twice a week) was administered intraperitoneally for mice of aflibercept treatment group for a week. The day on which treatment was started was designated as day 1. Tumor tissues were collected on day 8 and RNA-seq was conducted as described in Example 1.

### Results:

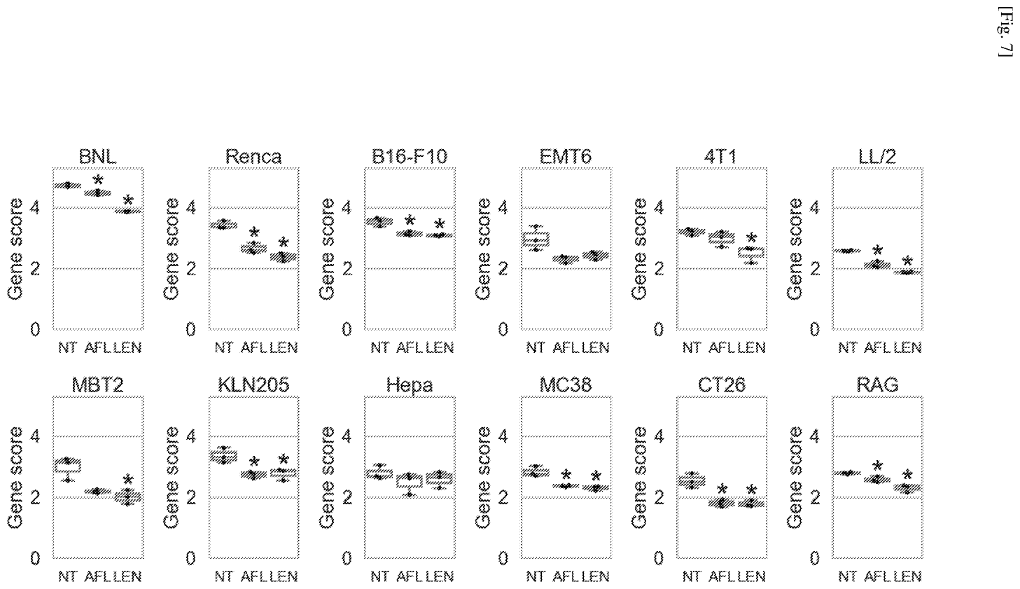

To investigate changes in gene score induced by lenvatinib treatment, gene score was calculated using RNA-seq data of tumor samples from non-treatment, aflibercept treatment, and lenvatinib treatment groups across 12 tumor models (FIG. 7). Gene score was significantly decreased in aflibercept treatment group compared with non-treatment group in eight tumor models (p<0.05, t-test). Gene score was decreased in lenvatinib treatment group in 10 tumor models (p<0.05, t-test). This result suggested that decrease of gene score was induced by both aflibercept and lenvatinib across tumor models, which is regardless of the sensitivity of tumors to lenvatinib. This result also suggested that gene score is a useful measure to infer anti-angiogenesis activity of angiogenesis inhibitors.

### Specific Embodiments

Specific embodiments of the invention are as follows:
(1) A method for treating a tumor in a human subject in need thereof, the method comprising:
quantifying mRNA expression level of selected genes in a tumor tissue obtained from the human subject, wherein the selected genes comprise at least two of kinase insert domain receptor (KDR), cadherin 5 (CDH5), endoglin (ENG), TEK receptor tyrosine kinase (TEK), protocadherin 12 (PCDH12) or SPARC like 1 (SPARCL1);
calculating a gene score based on the mRNA expression level of the selected genes quantified in the tumor tissue, wherein the gene score is higher than a control value; and
administering a angiogenesis inhibitor to the human subject,
wherein the tumor is not gastrointestinal cancer.
(2) The method of (1), wherein the tumor is thyroid cancer, renal cell carcinoma, breast cancer, endometrial cancer, glioblastoma, head and neck cancer, non-small cell lung cancer, ovarian cancer, pancreatic cancer, cervical cancer, hepatocellular carcinoma, thymic carcinoma, melanoma, urothelial carcinoma, colorectal cancer or biliary tract cancer.
(3) The method of (2), wherein the tumor is thyroid cancer, renal cell carcinoma, breast cancer, endometrial cancer, glioblastoma, head and neck cancer, non-small cell lung cancer, ovarian cancer, pancreatic cancer, cervical cancer or melanoma.
(4) The method of any one of (1) to (3), wherein the selected genes comprise at least three of KDR, CDH5, ENG, TEK, PCDH12 or SPARCL1.
(5) The method of any one of (1) to (3), wherein the selected genes comprise at least four of KDR, CDH5, ENG, TEK, PCDH12 or SPARCL1.
(6) The method of any one of (1) to (3), wherein the selected genes comprise at least five of KDR, CDH5, ENG, TEK, PCDH12 or SPARCL1.
(7) The method of any one of (1) to (3), wherein the selected genes comprise all of KDR, CDH5, ENG, TEK, PCDH12 and SPARCL1.
(8) The method of any one of (1) to (6), wherein the selected genes do not include KDR.
(9) The method of any one of (1) to (6), wherein the selected genes do not include CDH5.
(10) The method of any one of (1) to (6), wherein the selected genes do not include ENG.
(11) The method of any one of (1) to (6), wherein the selected genes do not include TEK.
(12) The method of any one of (1) to (6), wherein the selected genes do not include PCDH12.
(13) The method of any one of (1) to (6), wherein the selected genes do not include SPARCL1.
(14) The method of any one of (1) to (13), wherein the selected genes further comprise at least one gene other than KDR, CDH5, ENG, TEK, PCDH12 or SPARCL1.
(15) The method of any one of (1) to (13), wherein the selected genes do not comprise any gene other than KDR, CDH5, ENG, TEK, PCDH12 or SPARCL1.
(16) The method of any one of (1) to (15), wherein the tumor tissue has been obtained from a human subject before the administration of an angiogenesis inhibitor.
(17) The method of any one of (1) to (16), wherein the control value is a pre-determined cut-off value.
(18) The method of any one of (1) to (17), wherein the gene score is proportional to the average of logarithm-transformed mRNA expression level of the selected genes without weighting coefficient of each gene.
(19) The method of any one of (1) to (17), wherein the gene score is proportional to the average of logarithm-transformed mRNA expression level of the selected genes with weighting coefficients optimized for each gene.
(20) The method of any one of (1) to (19), wherein the angiogenesis inhibitor is a VEGF inhibitor.
(21) The method of (20), wherein the VEGF inhibitor is aflibercept or lenvatinib or a pharmaceutically acceptable salt thereof.
(22) The method of (21), wherein the lenvatinib or a pharmaceutically acceptable salt thereof is lenvatinib mesylate.
(23) A method of assessing microvessel density of a tumor tissue sample, the method comprising:
quantifying mRNA expression level of selected genes in a tumor tissue obtained from the human subject, wherein the selected genes comprise at least two of kinase insert domain receptor (KDR), cadherin 5 (CDH5), endoglin (ENG), TEK receptor tyrosine kinase (TEK), protocadherin 12 (PCDH12) or SPARC like 1 (SPARCL1); and
determining a gene score calculated from the mRNA expression level.
(24) The method of any one of (23), wherein the gene score is proportional to the average of logarithm-transformed mRNA expression level of the selected genes without weighting coefficient of each gene.
(25) The method of any one of (24), wherein the gene score is proportional to the average of logarithm-transformed mRNA expression level of the selected genes with weighting coefficients optimized for each gene.
(26) A kit comprising a container and a package insert,
wherein the container comprises an agent or agents for quantifying mRNA expression level of selected genes in a tumor tissue obtained from a human subject,
wherein the selected genes comprise at least two of kinase insert domain receptor (KDR), cadherin 5 (CDH5), endoglin (ENG), TEK receptor tyrosine kinase (TEK), protocadherin 12 (PCDH12) or SPARC like 1 (SPARCL1), and
wherein the package insert comprised instructions for calculating a gene score based on the mRNA expression level of selected genes comprising two or more of KDR, CDH5, ENG, TEK, PCDH12 or SPARCL1.
(1a) An angiogenesis inhibitor for use in a method for treating a tumor in a human subject;
wherein a gene score based on mRNA expression level of selected genes in a tumor tissue obtained from the human subject is higher than a control value;
wherein the selected genes comprise at least two of kinase insert domain receptor (KDR), cadherin 5 (CDH5), endoglin (ENG), TEK receptor tyrosine kinase (TEK), protocadherin 12 (PCDH12) or SPARC like 1 (SPARCL1); and
wherein the tumor is not gastrointestinal cancer.
(2a) The angiogenesis inhibitor of (1a), wherein the tumor is thyroid cancer, renal cell carcinoma, breast cancer, endometrial cancer, glioblastoma, head and neck cancer, non-small cell lung cancer, ovarian cancer, pancreatic cancer, cervical cancer, hepato-cellular carcinoma, thymic carcinoma, melanoma, urothelial carcinoma, colorectal cancer or biliary tract cancer.
(3a) The angiogenesis inhibitor of (2a), wherein the tumor is thyroid cancer, renal cell carcinoma, breast cancer, endometrial cancer, glioblastoma, head and neck cancer, non-small cell lung cancer, ovarian cancer, pancreatic cancer, cervical cancer or melanoma.
(4a) The angiogenesis inhibitor of any one of (1a) to (3a), wherein the selected genes comprise at least three of KDR, CDH5, ENG, TEK, PCDH12 or SPARCL1.
(5a) The angiogenesis inhibitor of any one of (1a) to (3a), wherein the selected genes comprise at least four of KDR, CDH5, ENG, TEK, PCDH12 or SPARCL1.
(6a) The angiogenesis inhibitor of any one of (1a) to (3a), wherein the selected genes comprise at least five of KDR, CDH5, ENG, TEK, PCDH12 or SPARCL1.
(7a) The angiogenesis inhibitor of any one of (1a) to (3a), wherein the selected genes comprise all of KDR, CDH5, ENG, TEK, PCDH12 and SPARCL1.
(8a) The angiogenesis inhibitor of any one of (1a) to (6a), wherein the selected genes do not include KDR.
(9a) The angiogenesis inhibitor of any one of (1a) to (6a), wherein the selected genes do not include CDH5.
(10a) The angiogenesis inhibitor of any one of (1a) to (6a), wherein the selected genes do not include ENG.
(11a) The angiogenesis inhibitor of any one of (1a) to (6a), wherein the selected genes do not include TEK.
(12a) The angiogenesis inhibitor of any one of (1a) to (6a), wherein the selected genes do not include PCDH12.
(13a) The angiogenesis inhibitor of any one of (1a) to (6a), wherein the selected genes do not include SPARCL1.
(14a) The angiogenesis inhibitor of any one of (1a) to (13a), wherein the selected genes further comprise at least one gene other than KDR, CDH5, ENG, TEK, PCDH12 or SPARCL1.
(15a) The angiogenesis inhibitor of any one of (1a) to (13a), wherein the selected genes do not comprise any gene other than KDR, CDH5, ENG, TEK, PCDH12 or SPARCL1.
(16a) The angiogenesis inhibitor of any one of (1a) to (15a), wherein the tumor tissue has been obtained from a human subject before the administration of an angiogenesis inhibitor.
(17a) The angiogenesis inhibitor of any one of (1a) to (16a), wherein the control value is a pre-determined cut-off value.
(18a) The angiogenesis inhibitor of any one of (1a) to (17a), wherein the gene score is proportional to the average of logarithm-transformed mRNA expression level of the selected genes without weighting coefficient of each gene.
(19a) The angiogenesis inhibitor of any one of (1a) to (17a), wherein the gene score is proportional to the average of logarithm-transformed mRNA expression level of the selected genes with weighting coefficients optimized for each gene.
(20a) The angiogenesis inhibitor of any one of (1a) to (19a), which is a VEGF inhibitor.
(21a) The VEGF inhibitor of (20a), which is aflibercept or lenvatinib or a pharmaceutically acceptable salt thereof.
(22a) The lenvatinib or a pharmaceutically acceptable salt thereof of (21a), which is lenvatinib mesylate.
(1b) Use of an angiogenesis inhibitor for the manufacture of a pharmaceutical composition for treatment of a tumor in a human subject;
wherein a gene score based on mRNA expression level of selected genes in a tumor tissue obtained from the human subject is higher than a control value;
wherein the selected genes comprise at least two of kinase insert domain receptor (KDR), cadherin 5 (CDH5), endoglin (ENG), TEK receptor tyrosine kinase (TEK), protocadherin 12 (PCDH12) or SPARC like 1 (SPARCL1); and
wherein the tumor is not gastrointestinal cancer.
(2b) The use of (1b), wherein the tumor is thyroid cancer, renal cell carcinoma, breast cancer, endometrial cancer, glioblastoma, head and neck cancer, non-small cell lung cancer, ovarian cancer, pancreatic cancer, cervical cancer, hepatocellular carcinoma, thymic carcinoma, melanoma, urothelial carcinoma, colorectal cancer or biliary tract cancer.
(3b) The use of (2b), wherein the tumor is thyroid cancer, renal cell carcinoma, breast cancer, endometrial cancer, glioblastoma, head and neck cancer, non-small cell lung cancer, ovarian cancer, pancreatic cancer, cervical cancer or melanoma.
(4b) The use of any one of (1b) to (3b), wherein the selected genes comprise at least three of KDR, CDH5, ENG, TEK, PCDH12 or SPARCL1.
(5b) The use of any one of (1b) to (3b), wherein the selected genes comprise at least four of KDR, CDH5, ENG, TEK, PCDH12 or SPARCL1.
(6b) The use of any one of (1b) to (3b), wherein the selected genes comprise at least five of KDR, CDH5, ENG, TEK, PCDH12 or SPARCL1.
(7b) The use of any one of (1b) to (3b), wherein the selected genes comprise all of KDR, CDH5, ENG, TEK, PCDH12 and SPARCL1.
(8b) The use of any one of (1b) to (6b), wherein the selected genes do not include KDR.
(9b) The use of any one of (1b) to (6b), wherein the selected genes do not include CDH5.
(10b) The use of any one of (1b) to (6b), wherein the selected genes do not include ENG.
(11b) The use of any one of (1b) to (6b), wherein the selected genes do not include TEK.
(12b) The use of any one of (1b) to (6b), wherein the selected genes do not include PCDH12.
(13b) The use of any one of (1b) to (6b), wherein the selected genes do not include SPARCL1.
(14b) The use of any one of (1b) to (13b), wherein the selected genes further comprise at least one gene other than KDR, CDH5, ENG, TEK, PCDH12 or SPARCL1.
(15b) The use of any one of (1b) to (13b), wherein the selected genes do not comprise any gene other than KDR, CDH5, ENG, TEK, PCDH12 or SPARCL1.
(16b) The use of any one of (1b) to (15b), wherein the tumor tissue has been obtained from a human subject before the administration of an angiogenesis inhibitor.
(17b) The use of any one of (1b) to (16b), wherein the control value is a pre-determined cut-off value.
(18b) The use of any one of (1b) to (17b), wherein the gene score is proportional to the average of logarithm-transformed mRNA expression level of the selected genes without weighting coefficient of each gene.
(19b) The use of any one of (1b) to (17b), wherein the gene score is proportional to the average of logarithm-transformed mRNA expression level of the selected genes with weighting coefficients optimized for each gene.
(20b) The use of any one of (1b) to (19b), wherein the angiogenesis inhibitor is a VEGF inhibitor.
(21b) The use (20b), wherein the VEGF inhibitor is aflibercept or lenvatinib or a pharmaceutically acceptable salt thereof.
(22b) The use of (21b), wherein the lenvatinib or a pharmaceutically acceptable salt thereof is lenvatinib mesylate.
(1c) A pharmaceutical composition for treating a tumor comprising an angiogenesis inhibitor;
wherein a gene score based on mRNA expression level of selected genes in a tumor tissue to be treated is higher than a control value;
wherein the selected genes comprise at least two of kinase insert domain receptor (KDR), cadherin 5 (CDH5), endoglin (ENG), TEK receptor tyrosine kinase (TEK), protocadherin 12 (PCDH12) or SPARC like 1 (SPARCL1); and
wherein the tumor is not gastrointestinal cancer.
(2c) The pharmaceutical composition of (1c), wherein the tumor is thyroid cancer, renal cell carcinoma, breast cancer, endometrial cancer, glioblastoma, head and neck cancer, non-small cell lung cancer, ovarian cancer, pancreatic cancer, cervical cancer, hepatocellular carcinoma, thymic carcinoma, melanoma, urothelial carcinoma, colorectal cancer or biliary tract cancer.
(3c) The pharmaceutical composition of (2c), wherein the tumor is thyroid cancer, renal cell carcinoma, breast cancer, endometrial cancer, glioblastoma, head and neck cancer, non-small cell lung cancer, ovarian cancer, pancreatic cancer, cervical cancer or melanoma.
(4c) The pharmaceutical composition of any one of (1c) to (3c), wherein the selected genes comprise at least three of KDR, CDH5, ENG, TEK, PCDH12 or SPARCL1.
(5c) The pharmaceutical composition of any one of (1c) to (3c), wherein the selected genes comprise at least four of KDR, CDH5, ENG, TEK, PCDH12 or SPARCL1.
(6c) The pharmaceutical composition of any one of (1c) to (3c), wherein the selected genes comprise at least five of KDR, CDH5, ENG, TEK, PCDH12 or SPARCL1.
(7c) The pharmaceutical composition of any one of (1c) to (3c), wherein the selected genes comprise all of KDR, CDH5, ENG, TEK, PCDH12 and SPARCL1.
(8c) The pharmaceutical composition of any one of (1c) to (6c), wherein the selected genes do not include KDR.
(9c) The pharmaceutical composition of any one of (1c) to (6c), wherein the selected genes do not include CDH5.
(10c) The pharmaceutical composition of any one of (1c) to (6c), wherein the selected genes do not include ENG.
(11c) The pharmaceutical composition of any one of (1c) to (6c), wherein the selected genes do not include TEK.
(12c) The pharmaceutical composition of any one of (1c) to (6c), wherein the selected genes do not include PCDH12.
(13c) The pharmaceutical composition of any one of (1c) to (6c), wherein the selected genes do not include SPARCL1.
(14c) The pharmaceutical composition of any one of (1c) to (13c), wherein the selected genes further comprise at least one gene other than KDR, CDH5, ENG, TEK, PCDH12 or SPARCL1.
(15c) The pharmaceutical composition of any one of (1c) to (13c), wherein the selected genes do not comprise any gene other than KDR, CDH5, ENG, TEK, PCDH12 or SPARCL1.
(16c) The pharmaceutical composition of any one of (1c) to (15c), wherein the tumor tissue has been obtained from a human subject before a treatment of an angiogenesis inhibitor.
(17c) The pharmaceutical composition of any one of (1c) to (16c), wherein the control value is a pre-determined cut-off value.
(18c) The pharmaceutical composition of any one of (1c) to (17c), wherein the gene score is proportional to the average of logarithm-transformed mRNA expression level of the selected genes without weighting coefficient of each gene.
(19c) The pharmaceutical composition of any one of (1c) to (17c), wherein the gene score is proportional to the average of logarithm-transformed mRNA expression level of the selected genes with weighting coefficients optimized for each gene.
(20c) The pharmaceutical composition of any one of (1c) to (19c), wherein the angiogenesis inhibitor is a VEGF inhibitor.
(21c) The pharmaceutical composition of (20c), wherein the VEGF inhibitor is aflibercept or lenvatinib or a pharmaceutically acceptable salt thereof.
(22c) The pharmaceutical composition of (21c), wherein the lenvatinib or a pharmaceutically acceptable salt thereof is lenvatinib mesylate.
(1d) A method for assessing microvessel density of a tumor tissue obtained from a human subject, the method comprising:
quantifying mRNA expression level of selected genes in a tumor tissue sample obtained from a human subject, wherein the selected genes comprise at least two of kinase insert domain receptor (KDR), cadherin 5 (CDH5), endoglin (ENG), TEK receptor tyrosine kinase (TEK), protocadherin 12 (PCDH12) or SPARC like 1 (SPARCL1); and
determining a gene score calculated from the mRNA expression level of the selected genes quantified in the tumor tissue,
wherein the tumor is not gastrointestinal cancer.
(2d) The method of (1d), wherein the tumor is thyroid cancer, renal cell carcinoma, breast cancer, endometrial cancer, glioblastoma, head and neck cancer, non-small cell lung cancer, ovarian cancer, pancreatic cancer, cervical cancer, hepatocellular carcinoma, thymic carcinoma, melanoma, urothelial carcinoma, colorectal cancer or biliary tract cancer.
(3d) The method of (2d), wherein the tumor is thyroid cancer, renal cell carcinoma, breast cancer, endometrial cancer, glioblastoma, head and neck cancer, non-small cell lung cancer, ovarian cancer, pancreatic cancer, cervical cancer or melanoma.
(4d) The method of any one of (1d) to (3d), wherein the selected genes comprise at least three of KDR, CDH5, ENG, TEK, PCDH12 or SPARCL1.
(5d) The method of any one of (1d) to (3d), wherein the selected genes comprise at least four of KDR, CDH5, ENG, TEK, PCDH12 or SPARCL1.
(6d) The method of any one of (1d) to (3d), wherein the selected genes comprise at least five of KDR, CDH5, ENG, TEK, PCDH12 or SPARCL1.
(7d) The method of any one of (1d) to (3d), wherein the selected genes comprise all of KDR, CDH5, ENG, TEK, PCDH12 and SPARCL1.
(8d) The method of any one of (1d) to (6d), wherein the selected genes do not include KDR.
(9d) The method of any one of (1d) to (6d), wherein the selected genes do not include CDH5.
(10d) The method of any one of (1d) to (6d), wherein the selected genes do not include ENG.
(11d) The method of any one of (1d) to (6d), wherein the selected genes do not include TEK.
(12d) The method of any one of (1d) to (6d), wherein the selected genes do not include PCDH12.
(13d) The method of any one of (1d) to (6d), wherein the selected genes do not include SPARCL1.
(14d) The method of any one of (1d) to (13d), wherein the selected genes further comprise at least one gene other than KDR, CDH5, ENG, TEK, PCDH12 or SPARCL1.
(15d) The method of any one of (1d) to (13d), wherein the selected genes do not comprise any gene other than KDR, CDH5, ENG, TEK, PCDH12 or SPARCL1.
(16d) The method of any one of (1d) to (15d), wherein the tumor tissue is obtained from a human subject before a treatment of the tumor.
(17d) The method of any one of (1d) to (16d), wherein the method further comprising a step of comparing the gene score with a control value.
(18d) The method of (17d), wherein the control value is a pre-determined cut-off value.
(19d) The method of any one of (1d) to (18d), wherein the gene score is proportional to the average of logarithm-transformed mRNA expression level of the selected genes without weighting coefficient of each gene.
(20d) The method of any one of (1d) to (18d), wherein the gene score is proportional to the average of logarithm-transformed mRNA expression level of the selected genes with weighting coefficients optimized for each gene.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a tumor in a human subject in need thereof, the method comprising:
quantifying mRNA expression level of selected genes in a tumor tissue obtained from the human subject, wherein the selected genes comprise at least two of kinase insert domain receptor (KDR), cadherin 5 (CDH5), endoglin (ENG), TEK receptor tyrosine kinase (TEK), protocadherin 12 (PCDH12) or SPARC like 1 (SPARCL1);
calculating a gene score based on the mRNA expression level of the selected genes quantified in the tumor tissue, wherein the gene score is higher than a control value; and
administering an angiogenesis inhibitor to the human subject,
wherein the tumor is not gastrointestinal cancer.

2. The method of claim 1, wherein the tumor is thyroid cancer, renal cell carcinoma, breast cancer, endometrial cancer, glioblastoma, head and neck cancer, non-small cell lung cancer, ovarian cancer, pancreatic cancer, cervical cancer, hepatocellular carcinoma, thymic carcinoma, melanoma, urothelial carcinoma, colorectal cancer or biliary tract cancer.

3. The method of claim 1, wherein the tumor is thyroid cancer, renal cell carcinoma, breast cancer, endometrial cancer, glioblastoma, head and neck cancer, non-small cell lung cancer, ovarian cancer, pancreatic cancer, cervical cancer or melanoma.

4.-6. (canceled)

7. The method of claim 1, wherein the selected genes comprise all of KDR, CDH5, ENG, TEK, PCDH12 and SPARCL1.

8.-13. (canceled)

14. The method of claim 1, wherein the selected genes further comprise at least one gene other than KDR, CDH5, ENG, TEK, PCDH12 or SPARCL1.

15. The method of claim 1, wherein the selected genes do not comprise any gene other than KDR, CDH5, ENG, TEK, PCDH12 or SPARCL1.

16. The method of claim 1, wherein the tumor tissue has been obtained from the human subject before the administration of the angiogenesis inhibitor.

17. The method of claim 1, wherein the control value is a pre-determined cut-off value.

18. The method of claim 1, wherein the gene score is proportional to the average of logarithm-transformed mRNA expression level of the selected genes without weighting coefficient of each gene.

19. The method of claim 1, wherein the gene score is proportional to the average of logarithm-transformed mRNA expression level of the selected genes with weighting coefficients optimized for each gene.

20. The method of claim 1, wherein the angiogenesis inhibitor is lenvatinib or a pharmaceutically acceptable salt thereof.

21. The method of claim 20, wherein the lenvatinib or a pharmaceutically acceptable salt thereof is lenvatinib mesylate.

22. A method of assessing microvessel density of a tumor tissue sample obtained from a human subject, the method comprising:
quantifying mRNA expression level of selected genes in a tumor tissue sample obtained from a human subject, wherein the selected genes comprise at least two of kinase insert domain receptor (KDR), cadherin 5 (CDH5), endoglin (ENG), TEK receptor tyrosine kinase (TEK), protocadherin 12 (PCDH12) or SPARC like 1 (SPARCL1); and
determining a gene score calculated from the mRNA expression level of the selected genes quantified in the tumor tissue,
wherein the tumor is not gastrointestinal cancer.

23. The method of claim 22, wherein the tumor tissue sample is from thyroid cancer, renal cell carcinoma, breast cancer, endometrial cancer, glioblastoma, head and neck cancer, non-small cell lung cancer, ovarian cancer, pancreatic cancer, cervical cancer, hepatocellular carcinoma, thymic carcinoma, melanoma, urothelial carcinoma, colorectal cancer or biliary tract cancer.

24. The method of claim 22, wherein the tumor tissue sample is from thyroid cancer, renal cell carcinoma, breast cancer, endometrial cancer, glioblastoma, head and neck cancer, non-small cell lung cancer, ovarian cancer, pancreatic cancer, cervical cancer or melanoma.

25.-27. (canceled)

28. The method of claim 22, wherein the selected genes comprise all of KDR, CDH5, ENG, TEK, PCDH12 and SPARCL1.

29.-34. (canceled)

35. The method of claim 22, wherein the selected genes further comprise at least one gene other than KDR, CDH5, ENG, TEK, PCDH12 or SPARCL1.

36. The method of claim 22, wherein the selected genes do not comprise any gene other than KDR, CDH5, ENG, TEK, PCDH12 or SPARCL1.

37. The method of claim 22, wherein the gene score is proportional to the average of logarithm-transformed mRNA expression level of the selected genes without weighting coefficient of each gene.

38. The method of claim 22, wherein the gene score is proportional to the average of logarithm-transformed mRNA expression level of the selected genes with weighting coefficients optimized for each gene.

39. (canceled)
